SEQUENCE LISTING (19 sequences)

SEQ ID NO: 1 - protein, 6 aa, Homo sapiens
TAYYIH

SEQ ID NO: 2 - protein, 17 aa, Homo sapiens
YISCYNGATNYNQKFKG

SEQ ID NO: 3 - protein, 17 aa, Homo sapiens
YISSYNGATNYNQKFKG

SEQ ID NO: 4 - protein, 17 aa, Homo sapiens
YISVYNGATNYNQKFKG

SEQ ID NO: 5 - protein, 11 aa, Homo sapiens
RDYDYDVGMDY

SEQ ID NO: 6 - protein, 119 aa, Homo sapiens
QVQLVQSGAEVKKPGASVKISCKASGYSFTAYYIHWVKQAPGQGLEWIGYISSYNGATNYNQKFKGRVTFTTDTSTSTAYMELRSLRSDDTAVYYCARDYDYDVGMDYWGQGTLVTVSS

SEQ ID NO: 7 - protein, 119 aa, Homo sapiens
QVQLVQSGAEVKKPGASVKISCKASGYSFTAYYIHWVKQAPGQGLEWIGYISCYNGATNYNQKFKGRVTFTTDTSTSTAYMELRSLRSDDTAVYYCARDYDYDVGMDYWGQGTLVTVSS

SEQ ID NO: 8 - protein, 119 aa, Homo sapiens
QVQLVQSGAEVKKPGASVKISCKASGYSFTAYYIHWVKQAPGQGLEWIGYISVYNGATNYNQKFKGRVTFTTDTSTSTAYMELRSLRSDDTAVYYCARDYDYDVGMDYWGQGTLVTVSS

SEQ ID NO: 9 - protein, 15 aa, Homo sapiens
RASESVDNYGISFMK

SEQ ID NO: 10 - protein, 7 aa, Homo sapiens
AASNQGS

SEQ ID NO: 11 - protein, 12 aa, Homo sapiens
QQSKEVPWTFGG

SEQ ID NO: 12 - protein, 111 aa, Homo sapiens
DIVMTQSPDSLAVSLGERATISCRASESVDNYGISFMKWFQQKPGQPPKLLIYAASNQGSGVPDRFSGSGSGTDFTLTISSLQAEDVAVYYCQQSKEVPWTFGGGTKVEIK

SEQ ID NO: 13 - protein, 522 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVG

SEQ ID NO: 14 - protein, 191 aa, Homo sapiens
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYC

SEQ ID NO: 15 - protein, 154 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQN

SEQ ID NO: 16 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 17 - protein, 8 aa, Homo sapiens
QAVVSPGP

SEQ ID NO: 18 - protein, 8 aa, Homo sapiens
LISKIAIQ

SEQ ID NO: 19 - protein, 8 aa, Homo sapiens
DYKDDDDK

(19) United States
(12) Patent Application Publication
(10) Pub. No.: US 2016/0324961 A1
(43) Pub. Date: Nov. 10, 2016

(54) Methods for Treating Cancer with DLL4 Antagonists

(71) Applicant: OncoMed Pharmaceuticals, Inc., Redwood City, CA (US)

(72) Inventors: Robert Joseph STAGG, Moraga, CA (US); Steven Eugene BENNER, Seattle, WA (US); John LEWICKI, Los Gatos, CA (US); Timothy Charles HOEY, Hillsborough, CA (US)

(21) Appl. No.: 15/097,631

(22) Filed: Apr. 13, 2016

Related U.S. Application Data
(63) Continuation of application No. 13/885,249, filed on Aug. 16, 2013, now abandoned.
(60) Provisional application No. 61/413,787, filed on Nov. 15, 2010.

(51) Int. Cl. A61K 39/395; A61K 31/337; A61K 31/7068

(52) U.S. Cl. CPC A61K 39/3955; A61K 31/7068; A61K 31/337; A61K 2039/505

(57) ABSTRACT

The present invention provides methods for treating cancer. More particularly, the invention provides methods for treating cancer comprising administrating doses of a DLL4 antagonist.

### CROSS REFERENCE TO RELATED APPLICATIONS

This application is a continuation of U.S. Application Ser. No. 13/855,249, which is the National Stage of International Application No. PCT/US11/60773, filed Nov. 15, 2011, which claims the priority benefit of U.S. Provisional Application No. 61/413,787, filed Nov. 15, 2010, each of which is hereby incorporated by reference herein in its entirety.

### REFERENCE TO A SEQUENCE LISTING SUBMITTED ELECTRONICALLY VIA EFS-WEB

The content of the electronically submitted sequence listing (name: 22930790002_sequenceisting_ascii.txt, size: 14.5 kilobytes: and date of creation: Apr. 12, 2016) is herein incorporated by reference in its entirety.

### DESCRIPTION OF THE INVENTION

1. Field of Invention

The present invention relates to the field of treating cancer. More particularly, the invention provides methods for treating cancer comprising administering doses of a DLL4 antagonist.

2. Background

Cancer is one of the leading causes of mortality in the developed world, with over one million people diagnosed with cancer and 500,000 deaths per year in the U.S. alone. Overall it is estimated that more than 1 in 3 people will develop some form of cancer during their lifetime. There are more than 200 different types of cancer, four of which—breast, lung, colorectal, and prostate—account for over half of all new cancer cases (Jemal et al., 2009, Cancer J. Clin. 58225-249).

Increasingly, treatment or cancer has moved from the use of systemically acting Cytotoxic drugs to include more targeted therapies that hone in on the mechanisms that allow and support unregulated cell growth and survival. For example, tumor angiogenesis, the process by which a tumor establishes an independent blood supply, is a critical step for tumor growth. Thus, efforts to target tumor angiogenesis have emerged as an important strategy for the development of novel cancer therapeutics.

Under normal conditions signaling pathways connect extracellular signals to the nucleus, leading to the expression of genes that directly or indirectly control cell growth, cell differentiation, cell survival, and cell death. In a wide variety of cancers, signaling pathways are dysregulated and may be linked to tumor initiation and/or tumor progression. Signaling pathways implicated in human oncogenesis include, but are not limited to, the Notch pathway, the Ras-Raf-MEK-ERK or MAPK pathway, the PI3K-AKT pathway, the CDKN2A/CDK4 pathway, the Bel-2/TP53 pathway, and the Wnt pathway.

The Notch signaling pathway is a universally conserved signal transduction system. It is involved in cell fate determination during development including embryonic pattern formation and post-embryonic tissue maintenance. In addition, Notch signaling has been identified as a critical factor in the maintenance of hematopoietic stem cells (HSCs).

The Notch pathway has been linked to the pathogenesis of both hematologic and solid tumors and cancers. Numerous cellular functions and microenvironmental cues associated with tumorigenesis have been shown to be modulated by Notch pathway signaling, including cell proliferation, apoptosis, adhesion, and angiogenesis. (Leong et al., 2006, Blood, 107:2223-2233). In addition, Notch receptors and/or Notch ligands have been shown to play potential oncogenic roles in a number of human cancers, including acute myclogenous leukemia, B cell chronic lymphocytic leukemia, Hodgkin lymphoma, multiple myeloma, T cell acute lymphoblastic leukemia, brain cancer, breast cancer, cervical cancer, colon cancer, lung cancer, pancreatic cancer, prostate cancer, skin cancer, and melanoma. (Leong et al., 2006, Blood, 107:2223-2233; Nickoloff et al., 2003, Oncogene, 22:6598-6608). Thus, the Notch pathway has been identified as a potential target for cancer therapy.

Previous studies demonstrated that antibodies to the human Notch ligand Delta-like ligand 4 (DLL4) can inhibit the growth of tumors and decrease the percentage of cancer stem cells or tumor initiating cells in some xenograft tumors (Hoey et al., 2009, Cell Stem Cell, 5:168-177). Anti-DLL4 antibodies have been shown to enhance angiogenic sprouting and branching which leads to non-productive angiogenesis and decreased tumor growth (Hoey et al., 2009, Cell Stem Cell, 5:168-177; Noguera-Troise et al., 2006, Nature, 444:1032-1037). These findings suggest that targeting the Notch pathway, for example with DLL4 antagonists, could help eliminate not only the majority of non-tumorigenic cancer cells, but also the tumorigenic cancer stem cells responsible for the formation and recurrence of solid tumors.

### SUMMARY OF THE INVENTION

The present invention provides methods for treating cancer comprising administering a therapeutically effective amount of a DLL4 antagonist to a human subject. In one aspect the invention provides methods for treating cancer in a human patient comprising; (a) administering to the patient an initial dose of a DLL4 antagonist; and (b) administering to the patient at least one subsequent dose of the DLL4 antagonist. In some embodiments, the method for treating cancer in a human patient comprises: (a) administering to the patient an initial dose of a DLL4 antagonist; (b) administering to the patient at least two subsequent doses of the DLL4 antagonist at a first dosing frequency; and (c) administering to the patient at least one additional subsequent dose of the DLL4 antagonist at a second dosing frequency. In certain embodiments, the first subsequent dose is administered about one week after the initial dose. In other embodiments, the first subsequent dose is administered about two weeks after the initial dose. In other embodiments, the first subsequent dose is administered about three weeks after the initial dose. In other embodiments, the first subsequent dose is administered about four weeks after the initial dose. In some embodiments, the subsequent doses in (b) are administered at a dosing frequency of about once a week or less. In some embodiments, the subsequent doses in (b) are administered at a dosing frequency of about once every 2 weeks. In some embodiments, the subsequent doses in (c) are administered at a dosing frequency of about once every 2 weeks. In some embodiments, the subsequent doses in (c) are administered at a dosing frequency of about once every 3 weeks.

In another aspect, the present invention provides methods for treating cancer in a human patient comprising, administering to the patient an effective dose of a DLL4 antagonist according to an intermittent dosing strategy. In some embodiments, the intermittent dosing strategy comprises administering an initial dose of a DLL4 antagonist to the patient, followed by subsequent doses or maintenance doses of the DLL4 antagonist once every 2 weeks, once every 3 weeks, or once every 4 weeks.

In some embodiments, the subsequent doses are about the same amount (i.e., mg/kg) or less than the initial dose. In other embodiments, the subsequent doses are more than the initial dose. In some embodiments, the initial dose is about 1 mg/kg to about 20 mg/kg.

In some embodiments, the DLL4 antagonist is administered as a fixed dose. In some embodiments, the initial dose is 2000 mg or less. In some embodiments, the initial dose is 1500 mg or less. In some embodiments, the initial dose is 1000 mg or less. In some embodiments, the initial dose is 500 mg or less. In some embodiments, the subseqnent doses are 1500 mg or less. In some embodiments, the subsequent doses are 1000 mg or less. In some embodiments, the subsequent doses are 750 mg or less. In some embodiments, the subsequent doses are 500 mg or less.

In certain embodiments, the method for treating cancer in a human patient comprises administering to the patient an initial dose of DLL4 antagonist of at least about 10 mg/kg, and followed by one or more subsequent doses of about 10 mg/kg or less.

In certain embodiments, the method for treating cancer in a human patient comprises: (a) administering to the patient an initial dose of a DLL4 antagonist of at least about 10 mg/kg; (b) administering to the patient two subsequent doses of the DLL4 antagonist of about 10 mg/kg about once a week; and (c) administering to the patient additional subsequent doses of the DLL4 antagonist of about 10 mg/kg about once every 2 weeks.

In some embodiments, the method for treating cancer in a human patient comprises: (a) administering to the patient an initial dose of a DLL4 antagonist; (b) administering to the patient subsequent doses of the DLL4 antagonist at a dosing frequency sufficient to achieve and maintain a therapeutically effective level of the DLL4 antagonist in the patient.

In some embodiments, the method for treating cancer in a human patient comprises: (a) administering to the patient an initial dose of a DLL4 antagonist; (b) administering to the patient subsequent doses of the DLL4 antagonist at dosing frequency sufficient to achieve a mean serum trough level of at least about 50 μg/ml of the DLL4 antagonist. In some embodiments, the mean serum trough level is at least about 75 μg/ml. In some embodiments, the mean serum trough level is at least about 100 μg/ml. In some embodiments, the mean serum trough level is at least about 125 μg/ml. In some embodiments, the mean serum trough level is at least about 15 g/ml.

In another aspect of the present invention, provided are methods for reducing one or more side effects and/or toxicities that result from the administration of a DLL4 antagonist.

In another aspect of the present invention, provided are methods for increasing the therapeutic index of a DLL4 antagonist.

In any of the aspects and/or embodiments described herein, the administration may be by intravenous injection or intravenously. In some embodiments, the administration is by intravenous infusion.

In any of the aspects and/or embodiments described herein, the cancer is selected from the group consisting of: lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon cancer, melanoma, and head and neck cancer.

In any of the aspects and/or embodiments described herein, the DLL4 antagonist specifically binds human DLL4. In some embodiments, the DLL4 antagonist is an antibody that specifically binds the extracellular domain of human DLL4. In some embodiments, the DLL4 antagonist specifically binds an epitope within amino acids 27-217 of the extracellular domain of human DLL4 (SEQ ID NO:14). In some embodiments, the DLL4 antagonist binds an epitope comprising amino acids 66-73 (QAVVSPGP, SEQ ID NO:17) of human DLL4. In some embodiments, the DLL4 antagonist binds an epitope comprising amino aids 139-146 (LISKIAIQ, SEQ ID NO:18):of human DLL4. In some embodiments, the DLL4 antagonist binds an epitope comprising amino acids 66-73 (QAVVSPGP, SEQ ID NO:17) and amino acids 139-146 (LISKIAIQ, SEQ ID NO:18) of human DLL4. In some embodiments, the DLL4 antagonist binds human DLL4 with a dissociation constant (K_{D}) of about 10 nM to about 0.1 nM.

In certain embodiments, the DLL4 antagonist is an anti-DLL4 antibody. In certain embodiments, the DLL4 antagonist comprises a heavy chain CDRI comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDRI comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11). In certain embodiments, the DLL4 antagonist comprises a heavy chain variable region comprising the amino acids of SEQ ID NO:6. In certain embodiments, the DLL4 antagonist further comprises a light chain variable region comprising the amino acids of SEQ ID NO:12. In certain embodiments, the DLL4 antagonist comprises the same heavy and light chain amino acid sequences as an antibody encoded by a plasmid deposited with ATCC having deposit no. PTA-8425 or PTA-8427. In certain embodiments, the DLL4 antagonist comprises the heavy chain CDR amino acid sequences and the light chain CDR amino acid sequences that are contained in the 21M18 antibody produced by the hybridoma deposited with ATCC having deposit no. PTA-8670. In certain embodiments, the DLL4 antagonist is encoded by the plasmid having ATCC deposit no. PTA-8423 which was deposited with the American Type Culture Collection (ATCC), at 10801 University Boulevard, Manassas, Va., 20110, under the conditions of the Budapest Treaty on May 10, 2007. In certain embodiments, the DLL4 antagonist is encoded by the plasmid having ATCC deposit no. PTA-8427 which was deposited with the American Type Culture Collection (ATCC), at 10801 University Boulevard, Manassas, Va., 20110, under the conditions of the Budapest Treaty on May 10, 2007. In some embodiments, the DLL4 antagonist is the antibody produced by the hybridoma having ATCC deposit no. PTA-8670 which was deposited with the ATCC under the conditions of the Budapest Treaty on Sep. 28, 2007. In certain embodiments, the DLL4 antagonist competes for specific binding to human DLL4 with an antibody encoded by the plasmid deposited with ATCC having deposit no. PTA-8425 or PTA-8427.

In certain embodiments, the method for treating cancer in a human patient comprises: (a) administering to the patient an initial dose of a DLL4 antagonist of at least about 10 mg/kg: (b) administering to the patient two subsequent doses of the DLL4 antagonist of about 10 mg/kg about once a week; and (c) administering to the patient additional subsequent doses of the DLL4 antagonist of about 10 mg/kg about one every 2 weeks, wherein the DLL4 antagonist comprises a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

In certain embodiments, the method for treating cancer in a human patient comprises: (a) administering to the patient an initial dose of a DLL4 antagonist; (b) administering to the patient subsequent doses of the DLL4 antagonist that provide a mean serum trough level of at least about 50 μg/ml of the DLL4 antagonist, wherein the DLL4 antagonist comprises a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

In some embodiments, the methods described herein further comprise administering at least one additional therapeutic agent. In certain embodiments, the additional therapeutic agent is an anti-hypertensive agent. In certain embodiments, the additional therapeutic agent is a chemotherapeutic agent.

In any of the aspects and/or embodiments described herein, the methods may reduce one or more side effects that result from the administration of a DLL4 antagonist, either alone or in combination with at least one additional therapeutic agent. In any of the aspects and/of embodiments described herein, the methods may reduce one or more toxicities that result from the administration of a DLL4 antagonist, either alone or in combination with at least one additional therapeutic agent. In any of the aspects and/or embodiments described herein, the methods may increase the therapeutic index a DLL4 antagonist, either alone or in combination with at least one additional therapeutic agent.

### DESCRIPTION OF THE FIGURES

FIG. 1. 6 week pharmacokinetic study of patients administered OMP-2IM18 10 mg/kg every other week. Observed and predicted results are shown.

FIG. 2. 8 week pharmacokinetic study of patients administered OMP-2IM18 10 mg/kg every other week. Observed and predicted results are shown.

FIG. 3. Waterfall plot showing percentage change in target lesions by dose group.

FIG. 4. Effect of intermittent dosing of anti-DLL4 antibody in pancreatic xenograft model. PN8 pancreatic tumor cells were injected subcutaneously into NOD/SCID mice. Mice were treated with anti-DLL4 antibody (20 mg/kg) once to week, anti-DLL4 antibody (20 mg/kg) once every 2 weeks, anti-DLL4 antibody (20 mg/kg) once every 4 weeks, gemcitabine once a week, a combination of gemcitabine (20 mg/kg) once a week and anti-DLL4 antibody (20 mg/kg) once a week, a combination of gemcitabine (20 mg/kg) once a week and anti-DLL4 antibody (20 mg/kg) once every 2 weeks, a combination of gemcitabine (20 mg/kg) once a week and anti-DLL4 antibody (20 mg/kg) once every 4 weeks, or control antibody (20 mg/kg) once a week. Data is shown as tumor volume (mm3) over days post-treatment

FIGS. 5A and B. Effect of intermittent dosing of anti-DLL4 antibody on tumorigenicity in pancreatic xenograft model. FIG. 5A) A subset of the data from FIG. 4 and including mice treated with anti-DLL4 antibody (mg/kg) once a week. FIG. 5B) Tumor cells isolated from treated mice were injected into NOD/SCID mice and allowed to grow without treatment for 64 days.

FIG. 6. Effect of intermittent dosing of anti-DLL4 antibody in a pancreatic tumor recurrence model. PN8 pancreatic tumor cells were injected subcutaneously into NOD/SCID mice. Mice were treated with gemcitabine (70 mg/kg) and anti-DLL4 antibody (20 mg/kg or 5 mg/kg) or gemcitabine and control antibody for 4 weeks. Antibody treatment continued after gemcitabine treatment was stopped. Anit-Dll4 antibody was administered once a week, once every 2 weeks or once every 4 weeks as indicated. Data is shown as tumor volume (mm3) over days post-treatment.

### DETAILED DESCRIPTION OF THE INVENTION

### Definitions

To facilitate an understanding of the present invention, a number of terms and phrases are defined below.

The term “antationist” as used herein includes any molecule that partially or fully blocks, inhibits, or neutralizes the expression of or the biological activity of a target molecule disclosed herein. Such biological activity includes, but is not limited to, inhibition of tumor growth and/or inhibition of tumor metastasis. The term “antagonist” includes any molecule that partially or fully blocks, inhibits, or neutralizes a biological activity of the Notch pathway. Suitable antagonist molecules include, but are not limited to, antibodies or fragments thereof which bind Notch receptors or Notch ligands (e.g., DLL4).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site or antigen-binding site within the variable region(s) of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen recognition site of an antibody, and any other modified immunoglobulin molecule comprising an antigen recognition site so long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy chain constant domains referred to as alpha, deka, epsilon, gamma, and mu, respectively. The different classes of immunoglobulins have different and well known subunit structures and three-dimensional configurations. Antibodies can be naked or conjugated to other molecules including, but not limited to, toxins and radioisotopes.

The term “antibody fragment” refers to a portion of an intact antibody and as used herein refers to the antigenic determining variable regions or the antigen-binding site of an intact antibody. Examples of antibody fragments include, but are not limited to Fab, Fab′, F(ab′)2, and Fv fingments, linear antibodies single chain antibodies, and multispecific antibodies formed from antibody fragments.

The term “variable region” of an antibody refers to the variable region of the antibody light chain or the variable region of the antibody heavy chain, either alone or in combination. The variable regions of the heavy and light chain each consist of four framework regions connected by three complementarity determining regions (CDRs) also known as hypervariable regions. The CDRs in each chain are held together in close proximity by the framework regions and, with the CDRs from the other chain, contribute to the formation of the antigen-binding site of the antibody. There are at least two techniques for determining CDRs; (I) an approach based on cross-species sequence variability (i.e., Kabat et al., 1991. Sequences of Proteins of Immunological Interest, 5th Edition, National Institutes of Health, Bethesda Md.); and (2) an approach based on crystallographic studies of antigen-antibody complexes (Al-Lazikani et al.., 1997, J. Molec. Biol. 273;927-948). In addition, combinations of these two approaches are sometimes used in the art to determine CDRs.

The term “monoclonal antibody” refers to a homogeneous antibody population involved in the highly specific recognition and binding of a single antigenic determinant, or epitope. This is in contrast to polyclonal antibodies that typically include a mixture of different antibodies directed against different antigenic detcaminants. The term “monoclonal antibody” encompasses both intact and full-length monoclonal antibodies as well as antibody fragments (such as Fab, Fab′, F(ab)2, Fv fragments), single chain Fv (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, hybridoma production, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human murine) sequences.

The term “human antibody” means an antibody produced by a human or an antibody having an amino acid sequence corresponding to an antibody produced by a human made using any technique known in the art. This definition of a human antibody includes intact or full-length antibodies, and fragments thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired specificity, affinity, and/or capability while the constant regions are homologous to the sequences in antibodies derived from another species (usually human) to avoid eliciting an immune response in that species.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids (often referred to as “linear epitopes”) and noncontiguous amino acids juxtaposed by tertiary folding of a protein (often referred to as “conformation epitopes”). Epitopes formed from contiguous amino acids are typically retained upon protein denaturing, whereas epitopes formed by tertiary folding are lost upon protein denaturing. An epitope typically includes at least 3, and more usually, at least 5 or 8-10 amino acids in a unique spatial conformation.

The terms “specifically binds” or “specific binding” mean that a binding agent or an antibody reacts or associates more frequently, more rapidly, with greater duration, with greater affinity, or with some combination of the above to an epitope or protein than with alternative substances, including unrelated proteins. In certain embodiments, “specifically binds” means, for instance, that an antibody binds to a protein with a K_{D} of about 0.1 mM or less, but more usually less than about 1 μM. In certain embodiments, “specifically binds” means that an antibody binds to a protein at times with a K_{D} of at least about 0.1 μM or less, and at other times at least about 0.0 μM or less. Because of the sequence identity between homologous proteins in different species, specific binding can include an antibody that recognizes a particular protein such as DLL4 in more than one species (e.g., mouse DLL4 and human DLL4). It is understood that an antibody or binding moiety that specifically binds to as first target may or may not specifically bind to a second target. As such, “specific binding” does not necessarily require (although it can include) exclusive binding, i.e. binding to a single target. Thus, an antibody may, in certain embodiments, specifically bind to more than one target. In certain embodiments, the multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. By way of non-limiting example, a bispecific antibody may comprise one antigen-binding site that recognizes an epitope on a DLL4 protein, and further comprises a second, different antigen-binding site thin recognizes a different epitope on a second protein, such as Notch. Generally, but not necessarily, reference to binding means specific binding.

The term “polypeptide” or “peptide” or “protein” are used interchangeably berm and refer to polymers of amino acids of any length. The polymer may be linear or branched, it may comprise modified amino acids, and it may be interrupted by non-amino acids. The terms also encompass an amino acid polymer that has been modified naturally or by intervention: for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “polynucleotide” or “nucleic acid”, are used interchangeably herein and refer to polymers of nucleotides of any length, and include DNA and RNA. The nucleotides can be deoxyribonucleotldes, ribonucleotides, modified nucleotides or bases; and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”; substitution of one or more of the naturally occurring nucleotides with an analog; internucleotide modifications such as uncharged linkages (e.g., methyl phosphonates, phosphotriesters, phosphoamidates cabanates, etc.) and charged linkages (e.g., phosphorothioates, phosphorodithioates, etc.); pendant moieties, such as proteins (e.g., nucleases, toxins, antibodies, signal peptides, poly-L-lysine, etc.); intercalators (e.g., acridine, psoralen, etc.): chelators (e.g., metals, radioactive metals, boron, oxidative metals, etc.); alkylators; modified linkages (e.g., alpha anomeric nucleic acids, etc.); as well as unmodified forms of the polynucleotide(s). Further, any hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′—O-methyl-, 2′—O-allyl, 2′-fluoro- or 2′-azido-ribose, carbocyclie sugar analogs, alpha-anoretic sugars, epimeric sugars such as arabinose, xyloses ar lyxoses, pyranose sugars, furanose sugars, heptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR:2 (“amidate”), P(O)R, P(O)OR′, CO or CH2 (“formacetal”), in which each R or R′ is independently H or substituted or unsubstituted alkyl (1-20 C) optionally containing an ,ether (—O—) linkage, aryl, alkenyl, cycloalkyl, cycloalkenyl or araldyl. Not all linkages in a polynucleotide need be identical.

“Conditions of high stringency” may be identified by those that: (1) employ low ionic strength and high temperature for washing, for example 0.015M sodium chloride/0.00015M sodium citrate/0.1% sodium dodecyl sulfate at 50° C.; (2) employ during hybridization a denaturing agent, such as formamide, for example, 50% (v/v) formamide with 0.1% bovine serum albumin/0.1% ficoll/0.1% polyvinylpyrrolidone/50 mM sodium phosphate buffer at pH 6.5 with 750 mM sodium chloride, 75 mM sodium citrate at 42° C.; or (3) employ 50% formamide 5× SSC (0.75M NaCl, 0.075M sodium curate), 50 mM sodium phosphate (pH 6.8), 0.1% sodium pyrophosphate, 5× Denhardt's solution, sonicated salmon sperm DNA (5 μg/ml) 0.1% SDS, and 10% dextran sulfate at 42° C., with washes at 42° C. in 0:2× SSC (sodium chloride/sodium citrate) and 50% formamide 55° C., followed by a high-stringency wash consisting of 0.0× SSC contraining EDTA 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software are known in the art that may be used to obtain alignments of amino acid or nucleotide sequences. These include, but are not limited to, BLAST, ALIGN, Megalign, and BestFit. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequenee comparison algorithm or by visual inspection. In some embodiments, identity exists over a region of the sequences that is at least about 10, at least about 20, at least about 40-60 residues in length or any integral value therebetween. In some embodiments, identity exists over a longer region than 60-80 residues, such as at least about 90-100 residues, and in some embodiments the sequences are substantially identical over the full length of the sequences being compared, sueh as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains(e.g., alanine, valine, leucine, isoleucine proline, phenylalanine, methionine, tryptophan), beta-branched side chains (e.g., threonine, Valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen(s), i.e., the DLL4 protein to which the polypeptide or antibody binds. Methods of identifying nucleotide and amino acid conservative substitutions which do not eliminate antigen binding are well-known in the art.

The term “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, and DNA or RNA expression vectors encapsulated liposomes.

A polypeptide, antibody, polynucleotide vector, cell, of composition which is isolated” is a polypeptide, antibody, polynucleotide, vector, cell, or composition which is in a form not found in nature. Isolated polypeptides, antibodies, polynucleotides vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), more preferably at least 90% pure, more preferably at least 95% pure, more preferably at least 98% pure, more preferably at least 99% pure.

The terms “tumor” and “neoplasm” refer to any mass of tissue that results from excessive cell growth or proliferation, either benign (noncancerous) or malignant (cancerous) including pre-cancerous lesions.

The terms “cancer stem cell” or “CSC” or “tumor stern cell” or “tumor initiating cell” or “solid tumor stem cell” or “tumorigenic stem cell” are used interchangeably herein and refer to a population of cells from a solid tumor that: (1) have extensive proliferative capacity; 2) are Capable of asymmetric cell division to generate one or more kinds of differentiated progeny with reduced proliferative or developmental potential; and (3) are capable of symmetric cell divisions for self-renewal or self-maintenance. These properties confer on the “cancer stem cells” or “tumor initiating cells” the ability to form palpable tumors upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor. These properties of self-renewal and proliferation to generate all other tumor cells confer on cancer stem cells the ability to form palpable tumors upon serial transplantation into an immunocompromised host a mouse) compared to non-tumorigenic tumor cells, which are unable to form tumors upon serial transplantation. It has been observed that non-tumorigenic tumor cells may form a tumor upon primary transplantation into an immunocompromised host after obtaining the tumor cells from a solid tumor, but those non-tumorigenic tumor cells do not give rise to a tumor upon serial transplantation.

The term “subject” refers to any animal (e.g., a mammal) including, but not limited to, humans, non-human primates, canines, felines, rodents, and the like, which is to be the recipient of a particular treatment. Typically, the terms “subject” and “patient” are used interchangeably herein in reference to a human subject.

The phrase “pharmaceutically acceptable salt” refers to a salt of a compound that is pharmaceutically acceptable and that possesses the desired pharmacological activity of the parent compound.

The phrase “pharmaceutically acceptable excipient, carrier or adjuvant” refers to an excipient, carrier or adjuvant that can be administered to a subject, together with at least one antagonist or antibody of the present disclosure, and which does not destroy the pharmacological and/or biological activity thereof and is nontoxic when administered in doses sufficient to deliver a therapeutic amount of the antagonist.

The phrase “pharmaceutically acceptable vehicle” refers to a diluent, adjuvant, excipient, or carrier with which at least one antagonist or antibody of the present disclosure is administered.

The term “therapeutically effective amount” refers to an amount of an antibody, polypeptide, polynueleotide, small organic molecule, or other drug effective to “treat” a disease or disorder in a subject or mammal. In the case of cancer, the therapeutically effective amount of the drug (e.g., an antibody) can reduce the number of cancer cells; reduce the tumor size; inhibit and/or stop cancer cell infiltration into peripheral organs including, for example, the spread of cancer into soft tissue and bone; inhibit and/or stop tumor metastasis; inhibit and/or stop tumor growth; relieve to some extent one or more of the symptoms associated with the cancer; reduce morbidity and mortality; improve quality of life; decrease tumorigenicity, tumorgenic frequency, or tumorgenic capacity of a tumor; reduce the number or frequency of cancer stem cells in a tumor; differentiate tumorigenic cells to a non-tumorigenic state; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following; is reduction in the number of, or complete absence of, cancer or tumor cells; a reduction in the tumor Size; inhibition of, or an absence of, cancer or tumor cell infiltration into peripheral organs including, for example, the spread of tumor into soft tissue and bone; inhibition of or an absence of, tumor metastasis; inhibition of, or an absence of, tumor growth; relief of one or more symptoms associated with the specific cancer; reduced morbidity and mortality; improvement in quality of life; reduction in tumorigenicity, tumorgenic frequency, or tumorgenic capacity of a tumor; reduction in the number or frequency of cancer stem cells in a tumor; reduction in the number or frequency of tumor initiating cells in a tumor; differentiation of tumorigenic cells to a non-tumorigenic state; or some combination of these effects.

As used in the present disclosure and claims, the singular forms “a” “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided.

The term “and/or” as used in a phrase such as “A and/or B” herein is intended to include both “A and B,” “A or B,” “A” and “B,” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone),

### II. DLL4 Antagonists

The present invention provides Dili antagonists for use in methods for treating cancer.

In certain embodiments, the DLL4 antagonist specifically binds the extracellular domain of human DLL4. In some embodiments, the DLL4 antagonist is an antibody. In some embodiments, the DLL4 antagonist or antibody specifically binds an epitope within amino acids 27-217 oldie extracellular domain of human DLL4 (SEQ ID NO:14). In some embodiments, the DLL4 antagonist or antibody specifically binds an epitope formed by a combination of the N-terminal region of human DLL4 (SEQ ID NO:15) and the DSL region of human DLL4 (SEQ ID NO:16). In some embodiments, the DLL4 antagonist or antibody binds an epitope comprising amino acids 66-73 (QAVVSPGP; SEQ ID NO:17) of human DLL4. In some embodiments, the DLL4 antagonist or antibody binds an epitope comprising amino acids 139-146 (LISKIAIQ, SEQ ID NO:18) of human DLL4. In some embodiments, the DLL4 antagonist or antibody binds an epitope comprising amino acids 66-73 (QAVVSPGP; SEQ ID NO:17) and amino acids 139-146 (LISKIAIQ; SEQ ID NO:18) of human DLL4.

In certain embodiments, the DLL4 antagonist (e.g., an antibody) binds to human DLL4 with a dissociation constant (K_{D}) of about 1 μM or less, about 100 nM or less, about 40 nM or less, about 20 nM or less, about 10 nM or less, or about 1 nM or less. In certain embodiments, the DLL4 antagonist or antibody binds to human DLL4 with a K_{D} of about 40 nM or less, about 20 nM or less, about 10 nM or less, or about 1 nM or less. In certain embodiments, the DLL4 antagonist binds to human DLL4 with a K_{D} of about 1 nM. In certain embodiments,. the DLL4 antagonist binds to human DLL4 with a K_{D} of about 0.8 nM. In certain etribodinients, the DLL4 -antagonist binds to human DLL4 with a K_{D} of about 0.6 nM. In certain embodiments, the DLL4 antagonist binds to human DLL4 with a K_{D} of about 0.5 nM. In certain embodiments, the DLL4 antagonist binds to human DLL4 with a K_{D} of about 0.4 nM. In some embodiments, the K_{D} is measured by surface plasmon resonance. In some embodiments, the dissociation constant of the antagonist or antibody to DLL4 is the dissociation constant determined using a DLL4 fusion protein comprising a DLL4 extracellular domain (e.g., DLL4 ECD-Fc fusion protein) immobilized on a Biacore chip.

In certain embodiments, the DLL4 antagonist (e.g., an antibody) binds to DLL4 with a half maximal effective concentration (EC₅₀) of about 1 μM or less., about 100 nM or less, about 40 nM or less, about 20 nM or less, about 10 nM or less, or about 1 nM or less. In certain embodiments, the DLL4 antagonist or antibody binds to human DLL4 with an EC₅₀ of about 40 nM or less, about 20 nM or less, about 10 nM or less, or about 1 nM or less.

In certain embodiments, the DLL4 antagonist is a polypeptide. In certain embodiments, the DLL4 antagonist polypeptide is an antibody. In certain embodiments, the antibody is an IgG antibody. In some embodiments, the antibody is an IgG1 antibody. In some embodiments, the antibody is an IgG2 antibody. In certain embodiments, the antibody is a monoclonal antibody. In certain embodiments, the antibody is a humanized antibody. In certain ernbodiments, the antibody is a human antibody. In certain embodiments, the antibody is an antibody fragment comprising an antigen-binding site.

The DLL4 antagonists (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochennstry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well known in the art (se, e.g., Ausubel et al., Editors, 1994-present, Current Protocols in Molecular Biology, John Wiley & Sons, inc. New York, N.Y.).

In some embodiments, the specific binding of a DLL4 antagonist (e.g., an antibody) to human DLL4 may be determined using ELISA. An ELISA assay comprises preparing DLL4 antigen, coating wells of a 96 well microtiter plate with antigen, adding to the wells the DLL4 antagonist or antibody conjugated to a detectable compound such as an enzymatic substrate (e.g., horseradish peroxidase or alkaline phosphatase), incubating for a period of time and detecting. the presence of the binding agent or antibody. In some embodiments, the DLL4 antagonist or antibody is not conjugated to a detectable compound, but instead a second cortjugated antibody that recognizes the DLL4 antagonist or antibody is added to the well. In some embodiments, instead of coating the well with DLL4 antigen, the DLL4 antagonist or antibody can be coated on the well, antigen is added to the coated well and then a second antibody conjugated to a detectable compound is added. One of skill in the art would be knowledgeable as to the parameters that can be modified and/or optimized to increase the signal detected, as well as other variations of ELISAs that can be used (see, e.g., Ausubel et Editors, 1994-present, Current Protocols in Molecular Biology, John Wiley & Sons, Inc., New York, N.Y.).

The binding affinity of an antagonist or antibody to DLL4 and the on-off rate of an antibody-antigen interaction can be determined by competitive binding assays. In some embodiments, a competitive binding assay is a radioimmunoassay comprising the incubation of labeled antigen (e.g., ³H or ^{t25}I), or fragment or variant thereof, with the antibody of interest in the presence of increasing amounts of unlabeled antigen followed by the detection of the antibody bound to the labeled antigen. The affinity of the antibody for the antigen and the on-off rates can be determined from the data by Scatchard plot analysis. In some embodiments, Biacore kinetic analysis is used to determine the binding affinities and on-off rates of antagonists or antibodies that bind DLL4. Biacore kinetic analysiscomprises analyzing the binding and dissociation of antibodies from antigens (e.g., DLL4 proteins) that have been immobilized on the surface of a Biacore chip. In some embodiments, Biacore kinetic analyses can be used to study binding of different antibodies in qualitative epitope competition binding assays.

In some embodiments, the DLL4 antagonists are polyelonal antibodies. Polyclonal antibodies can be prepared by any known method. Polyclonal antibodies are prepared by immunizing an animal (e.g., a rabbit, rat, mouse, goat, donkey) by multiple subcutaneous or intraperitoneal injections of the relevant antigen (e.g., a purified peptide fragment, full-length recombinant protein, fusion protein, etc.). The antigen can be optionally conjugated to a carrier protein such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, asettes and the like, of the immunized animal. Polyclonal antibodies can be purified from serum or ascites according to standard methods in the art including, but not limited to, affinity chromatography, ion-exchange chromatography, gel electrophoresis, and dialysis.

In some embodiments, the DLL4 antagonists are monoclonal antibodies. Monoclonal antibodies can be prepared using hybridoma methods known to one of skill in the art (see e.g., Kohler and Milstein, 1975, Nature 256:495). Using the hybridoma method, a mouse, hamster, or other appropriate host animal, is immunized as described above to elicit lymphocytes to produce antibodies that will specifically bind the immunizing antigen. In some embodiments, lymphocytes are immunized in vitro. In some embodiments, the immunizing antigen (e.g., DLL4) is a human protein or a portion thereof. In some embodiments, the immunizing antigen (e.g., DLL4) is a mouse protein or a portion thereof. In some embodiments, the immunizing antigen is an extracellular domain of human DLL4. In some embodiments, the immunizina antigen is an extracellular domain of mouse DLL4. In some embodiments, a mouse is immunized with a human antigen. In some embodiments, a mouse is immunized with a mouse antigen.

Following immunization, lymphocytes are isolated and fused with a suitable myeloma cell using, for example, polyethylene glycol. The hybridoma cells are selected using specialized media as known in the art and unfused lymphocytes and myeloma cells do not survive the selection process. Hybridomas that produce monoclonal antibodies directed against a target antigen may be identified by a variety of techniques including, but not limited to, immunoprecipitation, immunoblotting, and in vitro binding assays (e.g., flow cytometty, enzyme-linked immunosorbent assay (HASA), radioimmunoassay (RIA). The hybridomas can be propagated either in in vitro culture using standard methods (J. W. Goding, 1996, Monoclonal Antibodies: Principles and Practice, 3rd Edition, Academic Press, San Diego, Calif.) or in viva ascites in a host animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are cloned into suitable expression vectors which produce the monoclonal antibodies when transfected into host cells such as E. coli, simian COS cells, Chinese banister ovary (CHO) cells, or myeloma cells that do not otherwise produce immutioglobulin proteins. In certain embodiments, recombinant monoclonal antibodies, or fragiments thereof, can be isolated from phage display libraries expressing variable domain regions or CDRs of a desired species (see e.g., McCafferty et al., 1990, Nature, 348:552-554; Clackson et al., 1991, Nature, 352:624-628; and Marks et al., 1991, J. Mol. Biol, 222:581-597).

The polynucleotide(s) encoding a monoclonal antibody can be modified, for example, by using recombinant DNA technology to generate alternative antibodies. In some embodiments, the constant domains of the light and heavy chains of, for example, a mouse monoclonal antibody can be substituted for those regions of, for example, a human antibody to generate a chimeric antibody or for a non-immunoglobulin polypeptide to generate a fusion antibody. In some embodiments, the constant regions are truncated or removed to generate the desired antibody fragment of a monoclonal antibody. In some embodiments, site-directed or high-density mutagenesis of the variable region can be used to optimize specificity, affinity, and/or other biological characteristics of a monoclonal antibody. In some embodiments, site-directed mutagenesis of the CDRs can be used to optimize specificity, affy, and/or other biological characteristics of a monoclonal antibody.

In some embodiments, the DLL4 antagonist is a humanized antibody. Typically, humanized antibodies are human immunoglobulins in which residues from the complementary determining regions (CDRs) are replaced by residues from CDRs of a non-human species (e.g., mouse, rat, rabbit, hamster) that have the desired specificity, affinity, and/or capability by methods known to one skilled in the art. In some embodiments, the Fv framework region residues of a human immunoglobulin are replaced with the corresponding framework region residues from a non-human immunoglobulin that has the desired specificity, affinity, and/or capability. In some embodiments, the humanized antibody is further modified by the substitution of additional residues either in the Fv framework renion and/or within the replaced non-human residues to refine and optimize antibody specificity, affinity, and/or capability. In general, the humanized antibody will comprise substantially all of at least one, and typically two or three, variable domains containing all, or substantially all, of the CDRs that correspond to the non-human immunoglobulin whereas all, or substantially all, of the framework regions are those of a human immunoglobulln consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they should be less antigenic and may reduce HAMA (human anti-mouse antibody) responses when administered to a human subject. One skilled in the art would be able to obtain a functional humanized antibody with reduced immunogenicity following known techniques (see, e.g., U.S. Pat. Nos. 5,225,539; 5,585,089; 5,693,761; and 5,693,762).

In some embodiments, the invention provides an antibody that specifically binds the extracellular domain of human DLL4, wherein the antibody comprises one, two, three, four, five, and/or six of the CDRs of antibodies 21M18, 21M18 H9L2, and/or 21M18 H7L2. These antibodies have been described in U.S. Pat. No. 7,750,124. Antibodies 21M18 H7L2 and 21M18 H9L2 are humanized farms of the marine 21M 18 antibody.

In certain embodiments, the invention provides a DLL4 antagonist wherein the antagonist is a DLL4 antibody that specifically binds an epitope within amino acids 27-217 of the extracellular domain of human DLL4, and wherein the antibody comprises: a heavy chain CDR1 comprising TAYYIH (SEQ NO: 1), a heavy chain CDR2 comprising YISCYNGATNYNQKFKG (SEQ ID NO:2), YISSYNGATNYNQKFKG (SEQ ID NO:3), or YISVYNGATNYNQKFKG (SEQ ID NO:4), and a heavy chain CDR3 comprising RDYDYDVGMDY (SEQ NO:5). In some embodiments, the antibody further comprises a light chain CDR1 comprising RASESVDNYGISFMK (SEQ ID NO:9), a light chain CDR2 comprising AASNQGS (SEQ ID NO:10), and a light chain CDR3 comprisine QQSKEVPWTFGG (SEQ ID NO:11). In some embodiments, the antibody comprises alight chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

In certain embodiments, the invention provides an antibody that specifically binds an epitope within amino acids 27-217 of the extracellular domain of human DLL4, wherein the antibody comprises a heavy chain variable region having at least about 80% sequence identity to SEQ ID NO:6, and/or a light chain variable region haying at least 80% sequence identity to SEQ ID NO:12, In certain embodiments, the antibody comprises a heavy chain variable region having at least about 85%, at least about 90%, at least about 95%, at least about 97%, or at least about 99% sequence identity to SEQ ID NO:6. In certain embodiments, the antibody comprises a light chain variable region having at least about 85%, at least about 90%, at least about 95%, at least about 97%, or at least about 99% sequence identity to SEQ ID NO:12. In certain embodiments, the antibody comprises a heavy chain variable region having at least about 95% sequence identity to SEQ ID NO:6, and/or a light chain variable region having at least about 95% sequence identity to SEQ ID NO:12. In certain embodiments, the antibody comprises a heavy chain variable region comprising SEQ ID NO:6, and/or a light chain variable region comprising SEQ ID NO:12. In certain embodiments, the antibody comprises a heavy chain variable region comprising SEQ ID NO:6 and a light chain variable region comprising SEQ ID NO:12.

In certain embodiments, the DLL4 antagonist (e.g., an antibody) binds to the same epitope that an antibody comprising the heavy chain variable region comprising SEQ ID NO:6, and/or a light chain variable region comprising SEQ ID NO:12 binds. In some embodiments, the DLL4 antagonist or antibody binds to the same epitope as antibody 21M18. In some embodiments, the DLL4 antagonist or antibody binds to the same epitope as antibody 21M 18 H7L2. In some embodiments, the DLL4 antagonist or antibody binds to the same epitope as antibody 21M18 H9L2.

In certain embodiments, the DLL4 antagonist (e.g., an antibody) competes for specific binding, to an extracellular domain of human DLL4 with an antibody, wherein the antibody comprises a heavy chain variable region comprising SEQ ID NO:6, and/or a light chain variable region comprising SEQ ID NO:12. In some embodiments, the DLL4 antagonist competes for specific binding to an extracellular domain of human. DLL4 with an antibody encoded by the plasmid deposited with ATCC having deposit no, PTA -8425. In some embodiments, the DLL 4 antagonist or antibody competes for specific binding to an extracellular domain of human DLL4 with an antibody encoded by the plasmid deposited with ATCC having deposit no. PTA-8427. In some embodiments, the DLL4 antagonist or antibody competes for specific binding to an extracellular domain of human DLL4 with an antibody produced by the hybridoma deposited with ATCC having deposit no. PTA-8670. In some embodiments, the DLL4 antagonist or antibody competes for specific binding to an epitope within amino acids 27-217 of the extracellular domain of human DLL4 in a competitive binding assay.

In certain embodiments, the DLL4 antagonist is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated (see, e.g., Cole et al., 1985, Monoclonal Antibodies and Cancer Therapy, Alan R. Liss, p. 77; Boomer et al.. 1991, J. inommol 147:86-95; and U.S. Pat. Nos. 5,750,373; 5,567,610; and 5,229,275).

In some embodiments, the human antibody can be selected from a phage library, where that phage library expresses human antibodies (Vaughan et al., 1996, Nature Biotechnology, 14:309-314; Sheets et al.. 1998, PNAS, 95:6157-6162; Hoogenboom and Winter, 1991,J Mol. Biol., 227:381; Marks et al., 1991, J. Mol. Biol.., 222:551). Alternatively, phage display technology can be used to produce human antibodies and antibody fragments in vitro, from immunoglobulin variable domain gene repertoires from tmimmunized donors. Techniques for the generation and use of antibody phage libraries are described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7, 264,963; and Rothe et al., 2008, J. Mol. Bio., 376: 1182-1200.

Once antibodies are identified, affinity maturation strategies known in the art, including but not limited to, chain shuffling (Marks et al., 1992, Bio/Technology, 10:779-783) and site-directed mutagenesis, may be employed to generate high affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production. This approach is described in U.S. Pat. Nos. 5,545,807; 5,545,806; 5,569,825; 5,625,126; 5,633.425; and 5,661,016.

In certain embodiments, the DDL4 antagonist is a bispecific antibody. Bispecific antibodies are capable of specifically recognizing and binding to at least two different epitopes. The different epitopes can either be within the same molecule or on different molecules. In some enmbodiments, the antibodies can specifically recognize and bind a first antigen target, (e.g., DLL4) as well as a second antigen target, such as an effector molecule on a leukocyte (e.g CD2, CD3, CD28, or B7) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen, such as DLL4. These antibodies possess an antigen-binding arm and an arm which binds a cytotoxic agent or a radionuclide chelator, such as EOTUBE, DPTA, DOTA, or TETA. In certain embodiments, the antibodies can be used to affect angiogenesis. In certain embodiments, the bispecific antibody specifically binds DLL4, as well as VEGF. In certain embodiments, the bispecific antibody specifically binds DLL4, as well as a second Notch ligand (e.g., Jaaged1 or jagged2), or at least one Notch receptor selected from the group consisting of Notch1, Notch2, Notch3, and Notch4.

Techniques for making bispecific antibodies are known by those skilled in the art, see for example, Milistein et al., 1983, Nature, 305:537-539; Brennan et al., 1985, Science, 229:81 Suresh et al, 1986, Methods in Enzymol., 121:120; Traunecker et at, 1991, EMBO J., 10:3655-3659; Shalaby et al., 1992, J. Exp. Med., 175:217-225; Kostelny al., 1992, J. Immunol., 148;1547-1553; Gruber et al., 1994, J. Immunol., 152:5368; and U.S. Pat. No. 5,731,168). Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies to DLL4 are multispecific.

In certain embodiments, the DDL4 antagonists (e.g., antibodies or other polypeptides) described herein may be monospecific. For example, in certain embodiments, each of the one or more antigen-binding sites that an antibody contains is capable of binding (or binds) a homologous epitope on DLL4.

In certain embodiments, the DLL4 antagonist is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv single chain Fy (scFv) fragments. Fab, Fv, and scFv antibody fragments can be expressed in, and secreted from, E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science. 246:1275-1281) to allow rapid and effective identification of monoclonal Fab fragments with the desired specificity for DLL4, or derivatives, fragments, analogs or homologs thereof. In some embodiments, antibody fragments are linear antibody fragments. In certain embodiments, antibody fragments are monospecific or bispecific. In certain embodiments, the DDL4 antagonist is a scFv, Various techniques can be used for the production of single-chain antibodies specific to DLL4 (see, e.g., U.S. Pat. No. 4,946,778).

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antlbody fragment or by incorporating the epitope into a peptide tag that is then fused to the antibody fragment at either end or in the middle (e.g., by DNA or peptide synthesis).

For the purposes of the present invention, it should be appreciated that modified antibodies, or fragments thereof, can comprise any type of variable region that provides for the association of the antibody with DLL4. In this regard, the variable region may be derived from any type of mammal that can be induced to mount a humoral response and aenerate immunoglobulins against a desired antigen (e.g., DLL4). As such, the variable region of the modified antibodies can be, for example, of human, murine, non-human primate (e.g., cynomolgus monkeys, macaques, etc.) or lapine origin. In some embodiments, both the variable and constant regions of the modified inummoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification. Although the CDRs may be derived from an antibody of the same class or even subclass as the antibody from which the homework regions are derived, it is envisaged that the CDRs will be derived from an antibody of a different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen binding site.

Alterations to the variable reaion notwithstanding, those skilled in the art will appreciate that the modified antibodies of this invention will comprise antibodies (e.g.. full-length antibodies or antigen-binding fragments thereof) in which at least a fraction of one or more of the constant region domains has been deleted or otherwise altered so as to provide desired biochemical characteristics, such as increased tumor localization, increased tumor penetration, reduced serum half-life or increased serum half-life when compared with an antibody of approximately the same immunogenicity comprising a native or unaltered constant region. In some embodiments, the constant region of the modified antibodies comprises a human constant region. Modifications to the constant region include additions, deletions, or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the entire CH2 domain has been removed (ΔCH2 constructs). In some embodiments, the omitted constant region domain is replaced by a short amino acid spacer (e.g., 10 aa residues) that provides some of the molecular flexibility typically imparted by the absent constant region.

In certain embodiments, the modified antibodies .aru engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fe. binding and thereby increase tumor localization and/or tumor penetration. Similarly, it may be desirable to simply delete the part of one or more constant region domains that control a specific effector function (e.g., complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind to a cell expressing a Fc receptor (FCR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated tarsi t cells by killer cells, lease of inflammatory mediators, placental transfer and control of immunoglobulin production.

In certain embodiments, the DLL4 antibodies provide for altered effector functions that, in turn affect the biological profile of the administered antibody. For example, in some embodiments, the deletion or inactivation (through point mutations or other means) of a constant region domain may reduce Fc receptor binding of the circulating modified antibody (e.g., DLL4 antibody) thereby increasing tumor localization and/or penetration. In other embodiments, the constant region modifications increase or reduce the serum half-life of the antibody. In some embodiments, the constant region is modified to eliminate disulfide linkages or oligosaccharide moieties allowing for enhanced tumor localization and/or penetration.

In certain embodiments, a DLL4 antibody does not have one or more effector functions. In some embodiments, the antibody has no antibody-dependent cellular cytoxicity (ADC) activity and/or no complement-dependent cytoxicity (CDC) activity. In certain embodiments, the antibody does not bind to an Fc receptor and/or coMpleMent factors. In certain enbodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids.

Thus, the present invention provides methods for generating an antibody that binds the extracellular domain of human DLL4. In some embodiments, the method for generating an antibody that binds DLL4 comprises using hybridoma techniques. In some embodiments, the method comprises using an extracellular domain of mouse DLL4 or human DLL4 as an immunizing antigen. In some embodiments, the method of generating an antibody that binds DLL4 comprises screening a human phage library. The present invention further provides methods of identifying an antibody that binds to DLL4. In some embodiments, the antibody is identified by screening for binding to DLL4 with flow cytometry (FACS). In some embodiments, the antibody is screened for binding to human DLL4. In some embodiments, the antibody is screened for binding to mouse DLL4. In some embodiments, the antibody is identified by screening for inhibition or blocking of DLL4-induced Notch activation. In some embodiments, the DLL4 is human DLL4. In some embodiments, the Notch is human Notch1, Notch2, Notch3, or Notch4.

In certain embodiments, the antibodies described herein are isolated. In certain embodiments, the antibodies described herein are substantially pure.

Certain anti-DLL4 antibodies have been described, for example, in U.S. Pat. No. 7,350,124, which is incorporated by reference herein in its entirety. Certain additional anti-DLL4 antibodies are described in, e.g., International Patent Publication Nos. WO 2008/091222 and WO 2008/0793326, and U.S. Patent Application Publication Nos. 2008/0014196; 200870175847; 2008/0181899; and 2008/0107648, each of which is incorporated by reference herein in its entirety.

In some embodiments of die present invention, the DLL4 antagonists are polypeptides. The polypeptides can be recombinant polypeptides natural polypepodes or synthetic polypeptides that bind an epitope comprising amino acids within the extracellular domain of human DLL4. In some embodiments, the polypeptides comprise an antibody or fragment thereof that binds an epitope within the extracellular domain of human DLL4, It will be recognized by those of skill in the art that some amino acid. sequences of a polypeptide can be varied without significant effect on the structure or function of the protein. Thus, the polypeptides further include variations of the polypeptides which show substantial binding activity to an epitope of the human DLL4 protein. In some embodiments, amino acid sequence variations of polypeptides include deletions, insertions, inversions, repeats, and/or type substitutions,

The polypeptides and variants thereof, can be further modified to eontain additional chemical moieties not normally part of the pdlypeptide. The derivatized moieties can improve the the biological half-life, or the absorption of the polypeptide. The moieties can also reduee or eliminate any undesirable side effects of the polypeptides and variants. An overview for such chemical moieties can be found in Remington: The Science and Practice of Pharmacy, 21st Edition, 2005, University of the Sciences in Philadelphia, Pa.

The isolated polypeptides described herein can be produced by any suitable method known in the art. Such methods range from direct protein synthesis methods to constructing a DNA sequence encoding isolated polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and by selecting those codons that are favored in the host celi in which the recombinant polypeptide of interest will be produced. Standard methods can be applied to synthesize a polynucleotide sequence encoding a polypeptide of interest. For example, a complete amino acid sequence can be used to construct a back-translated gene. Further, a DNA oligomer containing a nucleotide sequence coding for the particular polypeptide can be synthesized. For example, several small oligonucleotides coding for portions of the desired polypeptide can be synthesized and then ligated. The individual oligonucleotides typically contain 5′ and/or 3′ overhangs for complementary assembly.

Once assembled (by synthesis, site-directed nrutagenesis, or another method), the polynucleotide sequences encoding a particular polypeptide of interest can be inserted into an expression vector and operatively linked to an expression control sequence appropriate for expression of the polypeptide in a desired host. Proper assembly can be confirmed by nucleotide sequencing, restriction mapping, and/or expression of a biologically active polypeptide in a suitable host. As is well-known in the art, in order to obtain high expression levels of a transieckid gene in a host, the gene must be operatively linked to transcriptional and translational expression control sequences that are functional in the chosen expression host.

In certain embodiments, recombinant expression vectors are used to amplify and express DNA encoding DLL4 antagonists such as polypeptides or antibodies, or fragments thereof. For example, recombinant expression vectors can be replicable DNA constructs which have synthetic or cDNA-derived DNA fragments encoding a polypeptide chain of an anti-DLL4 antibody, or fragment thereof, operatively linked to suitable transcriptional or translational regulatory elements derived from mammalian, microbial, viral, or insect genes. A transcriptional unit generally comprises an assembly of (1) a regulatory element or elements having a role in gene expression, for example, transcriptional promoters and/or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can also be incorporated, DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems inclnde a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of an expression vector and control elements depends upon the choice of host. A wide variety of expression host/vector combinations can be employed. Useful expression vectors for eukaryotic hosts include, for example, vectors comprising expression control sequences from SV40, bovine papiloma virus, adenovirus and cytomegalovirus. Useful expression vectors for bacterial hosts include known bacterial plasmids, such as plasmids from E. coli,including pCR1, pBR322, pMB9 and their derivatives, and wider host range plasmids, such as M13 and other filamentous single-stranded DNA phages.

Suitable host cells for expression of a DLL4 antagonist patypeptide or antibody (or a DLL4 protein to use as an antigen) include prokaryotes, yeast, insect, or higher eukaryotic cells under the control of appropriate promoters, Prokaryotes include gram-negative or gram-positive organisms, for example, E. coli or Bacilli. Higher eukaryotic cells include established cell lines of mammalian origin as described below. Cell-free translation systems can also be employed.

Various mammalian or insect cell culture systems are used to express recombinant protein. Expression of recombinant proteins in mammalian cells may be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived) cell lines, and HEK-293 (human embryonic kidney-derived) cell lines and variants thereof, Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are well-known to those of skill in the art (see, e.g., Lockow and Summers, 1988, Bio/Technology, 6:47).

The proteins produced by a transformed host can be purified according to any suitable method. Such methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence and glutathione-S-transferase can be attached to the protein to allow easy purification by passage over an appropriate affinity column. Isolated proteins can be physically characterized using such techniques as proteolysis, high performance liquid chromatography (HPLC), nuclear magnetic resonance (NMR), and x-ray crystallography.

For example, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, hydroxyupatite media is employed, including but not limited. to, ceramic hydroxyapatite (CHT). In some embodiments, one or more reversed-phase HPLC steps employing hydrophobic RP-HPLC media, (e.g., silica gel having pendant methyl or other aliphatic groups), is employed to further purify a protein. Some or all of the foregoing purification steps, in various combinations, can be employed to provide a homogeneous recombinant protein.

In some embodiments, recombinant proteinproduced in bacterial culture is isolated, for example, by initial extraction from cell pellets, followed by one or more concentration, salting-out, aqueous ion exchange, or sizeexclusion chromatography steps. In certain embodiments, HPLC is employed for final purification steps. Microbial cells employed in expression of a recombinant protein can be disrupted by any convenient method, including freeze-thaw cycling., sonication, mechanical disruption, or use of cell lysing agents.

Methods known in the art for purifying antibodies and other proteins also include, for example, those described in U.S. Patent Application Pub. Nos. 200830312425; 2009./0187005 and U.S. Pat. No. 7,691,980.

In certain embodiments, the DLL4 antagonist is a polypeptide that is not art antibody. A variety of methods for identifying and producing non-antibody polypeptides that bind with high affinity to a protein target are known in the art. See, e.g., Skerra, 2007, Curr. Opin. Biotechnol., 8:295-304; Hosse et al.. 2006, Protein Science, 15:14-27 Gill et al., 2006, Curr. Opin. Biotechnol., 17:653-658; Nygren, 2008, FEBS J., 275:2668-76; and Skerra, 2008. FEBS J., 275:2677-83. In certain embodiments, phage display technology may be used to produce and/or identify a DLL4 antagonist polypeptide. In certain embodiments, the DLL4 antagonist polypeptide comprises a protein scaffold of a type selected from the group consisting of protein A, protein G, a lipocalin, a fibronectin domain, an ankyrin consensus repeat domain, and thioredoxin.

In certain embodiments, the DLL4 antagonists or antibodies can be used in any one of a number of conjugated (e.g., an immunoconjugate err radioconjugate) or non-conjugated forms. In certain embodiments, the antibodies are used in non-conjugated form to harness the subject's natural defense mechanisms including CDC and/or ADCC to eliminate malignant or cancerous cells.

In certain embodiments, the DLL4 antagonist (e.g., an antibody or polypeptide) is conjugated to a cytotoxic agent. In some embodiments, the cytotoxic agent is a chemotherapeutic agent including, but not limited to, methotrexate, adriamicin, doxorubicin, melphalan, mitomycin C. chlarambucil, daunorubicin or other intercalating agents. In some embodiments, the cytotoxic agent is a enzymatically active toxin of bacterial, fungal, plant, or animal origin, or fragments thereof, including but notlimited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins. Phytolaca americana proteins (PAPI, PAPII, and PAP-S), Momordica charantia inhibitor, curcin, crotin, Sapaonaria offcinalis inhibitor, gelatin, restrictocin, phenornycin, enomycin, and the tricothecenes. In certain embodiments, the cytotoxic agent is a radioactive isotope to produce a radioconjugate or a radioconjugated antibody. A variety of radionuclides are available for the production of radioconjugated antibodies including, but not limited to, ⁵⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹²⁷Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diaconiunithenzoyl)-ethylenediamine), diisocyanates (such as tolyene 2,6-thisocyanate), and his-active fluorine compounds (such as 1,5-difluoro-2,4-dinitrobenzene).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents.

### IlI. Polynucleotides

In certain embodiments, the invention encompasses polynucleotides comprising polynucleotides that encode a polypeptide that specifically binds an epitope comprising amino acids within the extracellular domain of human DLL4 or a fragment of such a polypeptide. The term “polynucleotides that encode a polypeptide” encompasses a polynucleotide which includes only coding sequences for the polypeptide as well as a polynucleotide which includes additional coding and/or non-coding sequences. For example, the invention provides a polynucleotide comprising a nucleic acid sequence that encodes an antibody to a human DLL4 or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotides comprise the coding sequence for the mature polypeptide fused in the same reading frame to a polynucleotide which aids, for example, in expression and secretion of a polypeptide from a host cell (e.g., as leader sequence which functions as a secretory sequence for controlling transport of a polypeptide from the cell). The polypeptide having a leader sequence is a preprotein and can have the leader sequence cleaved by the host cell to produce the mature form of the polypeptide. The polynucleotides can also encode for a proprotein which is the mature protein plus additional 5′ amino acid residues. A mature protein having a prosequence is as proprotein and is an inactive form of the protein. Once the prosequence is cleaved an active mature protein remains.

In certain embodiments, the polynucleotides comprise the coding sequence for the mature polypeptide fused in the same reading frame to a marker sequence that allows for, for example, purification and/or identification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g. COS-7 cells) is used. In some embodiments, the marker sequence is a FLAG-tag, a peptide of sequence DYKDDDDK (SEQ ID NO: 19) which can be used in conjunction with other affinity tags.

The present invention further relates to variants of the hereinabove described polynucleotides encoding, for example, fragments, analogs, and/or derivatives.

In certain embodiments, the present invention provides isolated polynucleotides comprising polynucleotides having a nucleotide sequence at least 80% identical, at least 85% identical, at least 90% identical, at least 95% identical, and in some embodiments, at least 96%, 97%, 98%, or 99% identical to a polynucleotide encoding a polypeptide comprising an antibody, or fragment thereof, described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, the polynucleotide variants contain alterations which do not produce any changes in the amino acid sequence. In some embodiments, polynucleotide variants contain “silent” substitutions due to the degeneracy of the genetic code. Polynucleotide variants can be produced for a variety of reasons, for example, to optimize codon expression for a particular host (e.g., change codons in the human mRNA to those preferred by a bacterial host such as E. coli).

In certain embodiments, the polynucleotides described herein are isolated. In certain embodiments, the polynucleotides described herein are substantially pure.

Vectors and cells comprising the polynucleotides described herein are also provided.

### IV. Methods of Use and Pharmaceutical Compositions

The present invention provides methods for treating cancer in a human patient using the DLL4 antagonists (e.g., antibodies) described herein. One aspect of the invention provides methods for treating cancer in a human patient comprises; (a) administering to the patient an initial dose of a DLL4 antagonist; and (b) administering to the patient at least one subsequent dose of the DLL4 antagonist. In some embodiments, the method for treating cancer in a human patient comprises: (a) administering to the patient an initial dose of a DLL4 antagonist; (b) administering to the patient at least two subsequent doses of the DLL4 antagonist at a first dosing frequency; and (c) administering to the patient at least one additional subsequent dose of the DLL4 antagonist at a second dosing frequency. Achieving higher blood levels of a DLL4 antagonist at earlier timepoints may lead to more patients with stabilized disease, partial responses, or complete responses. Regimens that allow for this include higher initial doses, followed by subsequent doses at reduced levels; higher initial doses and increased dosing frequency at early timepoints; and/or initial doses at increased dosing frequency.

According to the invention, the initial dose or doses is/are followed by subsequent doses of equal or smaller amounts of DLL4 antagonist at intervals sufficient to maintain the antagonist at or above an efficacious target level. In some embodiments, the initial dose may be referred to as a “loading dose”. In some embodiments, the subsequent doses may be referred to as “maintenance doses”. The intervals between doses may be, but are not limited to, 1 week or less, about 2 weeks, about: 3 weeks, or about 4 weeks. In some embodiment, the higher initial dose or an increased dosing frequency of administration in the early weeks of treatment has the advantage of increased efficacy by reaching a target serum drug concentration earlier in treatment.

In certain embodiments, the first subsequent dose is administered about one week after the initial dose. In other embodiments, the first subsequent dose is administered about two weeks after the initial dose. In other embodiments, the first subsequent dose is administered about three weeks after the initial dose. In other embodiments, the first subsequent dose is administered about four weeks after the initial dose. In some embodiments, the subsequent doses in (b) are administered at a dosing frequency of about once a week or less. In some embodiments, the subsequent doses in (b) are administered at a dosing frequency of about once every 2 weeks. In some embodiments, the subsequent doses in (c) are administered at a dosing frequency of about once every 2 weeks. In some embodiments, the subsequent doses in (c) are administered at a dosing frequency of about once every 3 weeks.

In some embodiments, the subsequent doses are about the same amount or less than the initial dose. In other embodiments, the subsequent doses are a greater amount than the initial dose. As is known by those of skill in the art, doses used will vary depending on the clinical goals to be achieved. In some embodiments, the initial dose is about 1 mg/kg to about 20mg/kg. In some embodiments, the initial dose is about 2, 3, 4, 5, 6, 7, 8, 9, 10, 11, 12, 13, 14, 15, 16, 17, 18, 19, or 20mg/kg. In certain embodiments, the initial dose is about 2.5 mg/kg. In certain embodiments, the initial dose is about 5 mg/kg. In certain embodiments, the initial dose is about 7.5 mg/kg. In certain embodiments, the initial dose is about 10 mg/kg. In certain embodiments, the initial dose is about 12.5 mg/kg. In certain embodiments, the initial dose is about 15 mg/kg. In certain embodiments, the initial dose is about 20 mg/kg. In some embodiments, the subsequent doses are about 2 mg/kg to about 15 mg/kg. In certain embodiments, the subsequent doses are about 2, 3, 4, 5, 6, 7, 8, 9, 10, 11, 12, 13, 14 or 15mg/kg. In certain embodiments, the subsequent doses are about 2.5mg/kg. In certain embodiments, the subsequent doses are about 5 mg/kg. In some embodiments, the subsequent doses are about 7.5mg/kg. In some embodiments, the subsequent doses are about 10 mg/kg. In some embodiments, the subsequent doses are about 12.5 mg/kg.

In some embodiments, the initial dose of the DLL4 antagonist is 10 mg/kg, 12.5 mg/kg, 15 mg/ml, or 20 mg/kg. In some embodiments, the subsequent doses are 10 mg/kg administered once a week or once every 2 weeks. In some embodiments, the first two subsequent doses are 10 mg/kg administered once a week and subsequent doses are 10 mg/kg administered once every 2 weeks.

In some embodiments, the method for treating cancer in a human patient comprises administering to the patient an initial dose of a DLL4 antagonist of about 10 mg/kg or less, and followed by one or more subsequent doses of about 10 mg/kg or less. In some embodiments, the method for treating cancer in a human patient comprises administering to the patient an initial dose of DLL4 antagonist of about 5 mg/kg or less, and followed by one or more subsequent doses of about 5 mg/kg or less.

In some embodiments, the method for treating cancer in a human patient comprises: (a) administering to the patient an initial dose of a DLL14 antagonist of at least about 10 mg/kg; (b) administering to the patient two subsequent doses of the DLL4 antagonist of about 10 mg/kg about once a week:, and (c) administering to the patient additional subsequent doses of the DLL4 antagonist of about 10 mg/kg about once every 2 weeks.

In some embodiments, the DLL4 antagonist is administered as a fixed dose. In some embodiments, the initial dose is 2000 mg or less. In some embodiments, the initial dose is 1500 mg less. In some embodiments, the initial dose is 1000 mg or less. In some embodiments, the initial dose is 500 mg or less. In some embodiments, the subsequent doses are 1500 mg or less. In some embodiments, the subsequent doses are 1000 mg or less. In some embodiments, the subsequent doses are 750 mg or less. In some embodiments, the subsequent doses are 500 mg or less.

As is known to those of skill in the art, administration of any therapeutic agent may lead to side effects and/or toxicities. In some cases, the side effects and/or toxicities are so severe as preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or i f possible, suffer from the unpleasant side effects associated with the therapeutic agent.

Thus, one aspect of the present invention is directed to methods of treating cancer in a patient comprising using an intermittent dosing strategy for administering a DLL4 antagonist, which may reduce side effects and/or toxicities associated with adminisnation of the DLL4 antagonist. In some embodiments, a method for treating cancer in a human patient compdses administering to the patient an effective dose of a DLL4 antagonist according to as intermittent dosing strategy. In some embodiments, a method for treating cancer in a human patient comprises administering to the patient an effective dose of a DLL4 antagonist according to an intermittent dosing strategy, and increasing the therapeutic index of the DLL4 antagonist. In some embodiments, the intermittent dosing strategy comprises administering an initial dose of a DLL4 antagonist to the patient, and administering subsequent doses of the DLL4 antagonist about once every 2 weeks. In some embodiments, the intermittent dosing strategy comprises administering an initial dose of a DLL4 antagonist to the patient, and administering subsequent doses of the DLL4 antagonist about once every 3 weeks. In some embodiments, the intemittent dosing strategy comprises administering an initial dose of a DLL4 antagonist to the patient, and administering subsequent doses of the DLL4 antagonist about once every 4 weeks.

In some embodiments, the subsequent doses in an intermittent dosing strategy are about the same amount or less than the initial dose. In other embodiments, the subsequent doses are a greater amount than the initial dose. As is known by those of skill in the art, doses used will vary depending on the clinical goals to be achieved. In some embodiments, the initial dose is about 1 mg/kg to about 20 mg/kg. In some embodiments, the initial dose is about 2, 3,4, 5, 6, 7, 8, 9, 10, 11, 12, 13, 14, 15, 16, 17, 18, 19, or 20 mg/kg. In certain embodiments, the initial dose is about 2.5 mg/kg. In certain embodiments, the initial dose is about 5 mg/kg. In certain embodiments, the initial dose is about 7.5 mg/kg. In certain embodiments, the initial dose is about 10 mg/kg. In certain embodiments, the initial dose is about 12.5 mg/kg. In certain embodiments, the initial dose is about 15 mg/kg. In certain embodiments, the initial dose is about 20 mg/kg. In some embodiments, the subsequent doses are about 2 mg/kg to about 15 mg/kg. In certain embodiments, the subsequent doses are about 2, 3, 4, 5, 6, 7, 8, 9, 10, 11, 12, 13, 14 or 15 mg/kg. In certain embodiments, the subsequent doses are about 2.5 mg/kg. In certain embodiments, the subsequent doses are about 5 mg/kg. In some embodiments, the subsequent doses are about 7.5 mg/kg. In some embodiments, the subsequent doses are about 10 mg/kg. In some embodiments, the subsequent doses are about 12.5 mg/kg.

In some embodiments, the intermittent dosing strategy comprises: (a) administering to the patient an initial dose of a DLL4 antagonist of about 2.5 mg/kg and (b) administering subsequent doses of about 2.5 mg/kg once every 3 weeks. In some embodiments, the intermittent dosing strategy comprises: (a) administering to the patient an initial dose of a DLL4 antagonist of about 5 mg/kg and (b) administering subsequent maintenance doses of about 5 mg/kg once every 3 weeks. In some embodiments, the intermittent dosing strategy comprises: (a) administering to the patient an initial dose of a DLL4 antagonist of about 2.5 mg/kg and (b) administering subsequent maintenance doses of about 2.5 mg/kg once every 4 weeks, in some embodiments, the intermittent dosing strategy comprises: (a) administering to the patient an initial dose of a DLL4 antagonist of about 5 mg/kg and (b) administering subsequent maintenance doses of about 5 mg/kg once every 4 weeks. In certain embodiments, the initial dose and the maintenance doses are different, for example, the initial dose is about 5 mg/kg and the subsequent doses are about 2.5 mg/kg. In certain embodiments, an intermittent dosing strategy may comprise a loading dose, for example, the initial dose is about 20 mg/kg and the subsequent doses are about 2.5 mg/kg or about 5 mg/kg administered once every 2 weeks, once every 3 weeks, or once every 4 weeks.

In some embodiments, a method for reducing toxicity of a DLL4 antagonist in a human patient comprises administering to the patient the DLL4 antagonist using an intermittent. dosing strategy. In some embodiments, a method for reducing side effects of a DLL4 antagonist in a human patient comprises administering to the patient the DLL4 antagonist using an intermittent dosing strategy. In some embodiments, a method for increasing the therapeutic index of a DLL4 antagonist in a human patient comprises administering to the patient the DLL4 antagonist using an intermittent dosing strategy.

In another aspect of the invention, provided are methods for treating cancer in a human patient comprising: (a) administering to the patient an initial dose of a DLL4 antagonist, and (b) administering to the patient subsequent doses of the DLL4 antagonist at a dosing frequency sufficient to achieve and maintain a therapeutically effective level of the DLL4 antagonist in the patient.

In another aspect of the invention, provided are methods for treating cancer m a human patient comprising: (a) administering to the patient an initial dose of a DLL4 antagonist, and (b) administering to the patient subsequent doses of the DLL4 antagonist at a dosing frequency sufficient to achieve a mean serum trough level of at least about 50 μg/ml of the DLL4 antagonist. In some embodiments, the mean serum trough level is at least about 75 μg/ml. In some embodiments, the mean serum trough level is at least about 100 μg/ml. In some embodiments, the mean serum trough level is at least about 125 μg/ml. In some embodiments, the mean serum trough level is at least about 150 μg/ml.

The choice of delivery method for the initial and subsequent doses is made according to the ability of the animal or human patient to tolerate introduction of the DLL4 antagonist into the body. Thus, in any of the aspects and/or embodiments described herein, the administration of the DLL4 antagonist may be by intravenous injection or intravenously. In some embodiments, the administration is by intravenous infusion. In any of the aspects and/or embodiments described herein, the administration of the DLL4 antagonist may be by a non-intravenous route.

In any of the aspects and/or embodiments described herein, provided are methods for treating cancer, wherein the cancer is selected from the group consisting of lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon cancer, melanoma, and head and neck cancer.

In any of the aspects andfor embodiments described herein, provided are methods for treating cancer by administering to the patient a DLL4 antagonist. In some embodiments, the DLL4 antanonist is an antibody that specifically binds the extracellular domain of human DLL4. In some embodiments, the DLL4 antagonist specifically binds an epitope within amino acids 27-217 of the extracellular domain of human DLL4 (SEQ ID NO:14). In some embodiments, the DLL4 antagonist binds an epi tope comprising amino acids 66-73 (QAVVSPGP, SEQ ID NO:17) of human DLL4. In some embodiments, the DLL4 antagonist binds an epitope comprising amino acids 139-146 (LISKIAIQ, SEQ NO:18) of human DLL4. In some embodiments, the DLL4 antagonist binds an epitope comprising amino acids 66-73 (QAVVSPGP, SEQ ID NO:17) and 139-146 (LISKIAIQ, SEQ ID NO:18) of human DLL4. In some embodiments, the DLL4 antagonist binds human DLL4 with a dissociation constant (K_{D}) of about 10 nM to about 0.1 nM or less.

In certain embodiments, the DLL4 antagonist is an anti-DLL4 antibody. In certain embodiments, the DLL14 antagonist comprises a heavy chain CDR1 comprising TAVYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11). In certain embodiments, the DLL4 antagonist comprises a heavy chain variable region comprising the amino acids of SEQ IL) NO:6. In certain embodiments, the DLL4 antagonist further comprises a light chain variable region comprising the amino acids of SEQ ID NO:12. In certain embodiments, the DLL4 antagonist comprises the same heavy and light chain amino acid sequences as an antibody encoded by a plasmid deposited with ATCC having deposit no. PTA-8425 or PTA-8427. In certain embodiments, the DLL4 antagonist comprises the heavy chain CDR amino acid sequences and the light chain CDR amino acid sequences that are contained in the 21M18 antibody produced by the hybridoma deposited with ATCC having deposit no. PTA-8670. In certain embodiments, the DLL4 antagonist is encoded by the plasmid having ATCC deposit no. PTA-8425 which was deposited with the American Type Culture Collection (ATCC), at 10801 University Boulevard, Manassas, Va., 20110, under the conditions of the Budapest Treaty on May 10, 2007. In certain embodiments, the DLL4 antagonist is encoded by the plasmid having ATCC deposit no. PTA-8427 which was deposited with the American Type Culture Collection (ATCC), at 10801 University Boulevard, Manassas, Va., 20110, under the conditions of the Budapest Treaty on May 10, 2007. In some embodiments, the DLL4 antagonist is the antibody produced by the hybridoma having ATCC deposit no. PTA-8670 which was deposited with the ATCC under the conditions of the Budapest Treaty on Sep. 28, 2007, In certain embodiments, the DLL4 antagonist competes for specific binding to human DLL4 with an antibody encoded by the plasmid deposited with ATCC having deposit no. PTA-8425 or PTA-4427.

In certain embodiments, the method for treating cancer in a human patient comprises: (a) administering to the patient an initial dose of a DLL4 antagonist of at least about 10 mg/kg; (b) administering to the patient two subsequent doses of the DLL4 antagonist of about 10 mg/kg about once a week; and (c) administering to the patient additional subsequent doses of the DLL4 antagonist of about 10 mg/kg, about once every 2 weeks, wherein the DLL4 antagonist comprises a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

In some embodiments, the method for treating cancer in a human patient comprises administering to the patient an effective dose of a DLL4 antagonist according to an intermittent dosing strategy, wherein the DLL4 antagonist comprises a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDRI comprising RASESVDNYGISFMK (SEQ ID NO:9), a light chain CDR2 comprising AASNQGS (SEQ ID NO:10), and a light chain CDR3 comprising QQSKEVPWTFGG (SEQ NO:11).

In certain embodiments, the method for treating cancer in a human patient comprises: (a) administering to the patient an initial dose of a DLL4 antagonist (h) administering to the patient subsequent doses of the DLL4 antagonist that provide a mean serum trough level of at least about 50 μg/ml of the DLL4 antagonist, wherein the DLL4 antagonist comprises a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light Chain CDR1. comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11),

In some embodiments, the method of treating cancer comprises administration of an initial dose of a DLL4 antagonist of about 10 mg/kg. For example, antibody OMP-21M18 is diluted with 5% dextrose in water (USP) to a total volume of 250 mL. The OMP-21M18 is delivered through a 0.22-micron filter over 30 minutes as an intravenous infusion. In some embodiments, subsequent doses are administered in a similar manner.

In another aspect of the invention, the methods described herein may further comprise administering at least one additional therapeutic agent. An additional therapeutic agent can be administered prior to, concurrently with, and/or subsequently to, administration of the DLL4 antagonist. Pharmaceutical compositions comprising a DLL4 antagonist and an additional therapeutic agent(s) are also provided. In some embodiments, the at least one additional therapeutic agent comprises 1, 2, 3, or more additional therapeutic agents.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing side effects and/or toxicities. Combination therapy may decrease the likelihood that resistant cancer cells will develop. In some embodiments, combination therapy comprises a therapeutic agent that primarily affects (e.g., inhibits or kills) non-tumorigenic cells and a therapeutic agent that primarily affects (e.g., inhibits or kills) tumorigenic CSCs.

It will be appreciated that the combination of a DLL4 antagonist and an additional therapeutic agent may be administered in any order or concurrently. In some embodiments, the DLL4 antagonist is administered to patients that have previously undergone treatment with a second therapeutic agent. In certain other embodiments, the DLL4 antagonist and a second therapeutic agent is administered substantially simultaneously or concurrently. For example, a subject may be given a DLL4 antagonist (e.g., an antibody) while undergoing a course of treatment with a second therapeutic agent (e.g., chemotherapy). In certain embodiments, a DLL4 antagonist is administered within 1 year of the treatment with a second therapeutic agent. In certain alternative embodiments, a DLL4 antagonist is administered within 10, 8, 6, 4, or 2 months of any treatment with a second therapeutic agent. In certain other embodiments, a DLL4 antagonist is administered within 4, 3, 2, or 1 weeks of any treatment with a second therapeutic aaent. In some embodiments, a DLL4 antagonist is administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

Useful classes of therapeutic agents include, for example, antitubulin agents, auristatins DNA minor groove binders, DNA replication inhibitors, alkylating agents (e.g., platinum complexes such as cisplatin, mono(platinurn), bis(platinum) and tri-nuclear platinum complexes and carboplatin), anthracyclines, antibiotics, antifolates, antimetabolites, chemotherapy sensitizers, duocarmycins, etoposides, fluorinated pyrinndines, ionophores, lexitropsins, nitiosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetaboiite, an antimitotic, topoisomerase inhibitor, or an angiogenesis inhibitor. In some embodiments, the second therapeutic agent is a platinum complex such as carboplatin cisplatin. In some embodiments, the additional therapeutic agent is a platinum complex in combination with a taxane. In certain embodiments, the additional therapeutic agent is an anti-hypertensive agent.

Therapeutic agents that may be administered in combination with the DLL4 antagonist include chemotherapeutic agents. Thus, in some embodiments, the method or treatment involves the administration of a DLL4 antagonist of the present invention in combination with a chemotherapeutic agent or cocktail of multiple different chemotherapeutic agents. Treatment with a DLL4 antagonist (e.g., an antibody) can occur prior to, concurrently with, or subsequent to administration of chemotherapies. Combined administration can include co-administration, either in a single pharmaceutical formulation or using separate foimulations, or consecutive administration in either order but generally within a time period such that all active agents can exert their biological activities simultaneously. Preparation and closing schedules for such chemotherapeutic agents can be used according to manufacturers instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclophosphamide (CYTOXAN); alkyl sulfonates such as busulfan, improsulfan and piposulfan; aziridines such as benzodopa carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelanayem rodorubicin, streptonigrin, streptozocin, tubercidin, abenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside: aminolevulinic acid; amsacrine; bestrabucil; bisantrene; edatraxate; defofamine; demecolcine; diaziquone; el formithine; elliptinium acetate; etoglucid; gallium nitrate; hydroxyurea; lentinan; lonidamine; mitoguazone; mitoxantrone; mopidamol; nitracrine; pentostatin; phenamet; pirarubicin: podophyllinic acid; 2-ethylltydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; nacytosine; arabinoside (Ara-C); taxoids e.g. paclitaxel (TAXOL) and docetaxel (TAXOTERE); chlorambucil; gemcitabine;6 -thioguanine; mercaptopurine; platinum analogs such as cisplatin and carboplatin; vinblastine; platinum; etoposide (VP-16); ifosfamide; mitomycin C; mitoxantrone; vincristine.; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA) and pharmaceutically acceptable salts, acids or derivatives of any of the above. Chemotherapeutic agents also include anti-hormonal agents that act to regulate or inhibit hormone action on tumors such as anti-estrogens including, for example, tamoxifen, raloxifene, aromatase inhibiting 4(5)-imidazoles, 4-hydroxytamoxifen, trioxifene, keoxifene, LYN117018, onapistone, and toremifene (FARESTON); and anti-androgens such as flutamide, nilutamide, bicalutamide, leuprolide, and goserelin: and pharmaceutically acceptable salts, acids or derivatives of any of the above. In certain embodiments, the additional therapeutic agent is cisplatin. In certain embodiments, the additional therapeutic agent is carboplatin. In certain embodiments, the additional thorapeutie agent is paclitaxel. In certain embodiments, where the chemotherapeutic agent administered in combination with a DLL4 antagonist is carboplatin, the cancer or tumor being treated is lung cancer or a lung tumor.

In certain embodiments, the chemotherapeutic agent is a topoisomerase inhibitor. Topoisomerase inhibitors are chemotherapeutic agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In certain embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, peruetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is nemeitabine. In some embodiments, the additional therapeutic agent is pemetresed. In certain embodiments, where the chemotherapeutic agent administered in combination with a DLL4 antagonist is gemcitabine, the cancer or tumor being treated is pancreatic cancer or a pancreatic tumor. In certain embodiments, where the chemotherapeutic agent administered in combination with a DLL4 antagonist is pemetrexed, the cancer or tumor being treated is lung cancer or a lung tumor.

In certain embodiments, the chemotherapeutic agent is an antimitotie agent, including, but not limited to, agents that bind tubulin. In some embodiments, the agent is a taxane. In certain embodiments, the agent is paclitaxel docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinea alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Pik1. In certain embodiments, where the chemotherapeutic agent administered in combination with a DLL4 antagonist is an anti-mitotic agent, the cancer or tumor being treated is breast cancer or a breast tumor.

In some embodiments, an additional therapeutic agent comprises an agent such as a small molecule. For example, treatment can the combined administration of a DLL4 antagonist (e.g., an antibody) of the present invention with a small molecule that acts as an inhibitor against additional tumor-associated proteins including, but not limited to, EGFR, ErbB2, HER2, and/or VEGF. In certain embodiments, the additional therapeutic agent is a small molecule that inhibits a cancer stem cell pathway. In some embodiments, the additional therapeutic agent is a small molecule inhibitor of the Notch pathway. In some embodiments, the additional therapeutic agent is a small molecule inhibitor of the Wnt pathway. In some embodiments, the additional therapeutic agent is a small molecule inhibitor of the BMP pathway. In some embodiments, the additional therapeutic agent is a small molecule that inhibits β-catenin signaling.

In some embodiments, an additional therapeutic agent comprises a biological molecule, such as an antibody. For example, treatment can involve the combined administration of a DLL4 antagonist (e.g., an antibody) of the present invention with other antibodies against additional tumor-associated proteins including, but not limited to, antibodies, that bind EGFR, ErbB2, HER2, and/or VEGF. In certain embodiments, the additional therapeutic agent is an antibody that is an anti-cancer stem cell marker antibody. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an antibody inhibitor of the Notch pathway. In some embodiments, the additional therapeutic agent is an antibody inhibitor of the Wnt pathway. In some embodiments, the additional therapeutic agent is an antibody inhibitor of the BMP pathway. In some embodiments, the additional therapeutic agent is an antibody that inhibits β-catenin signaling. In certain embodiments, the additional therapeutic agent is an antibody that is an angiogenesis inhibitor or modulator (e.g., an anti-VEGF or VEGF receptor antibody). In certain embodiments, the additional therapeutic agent is bevacizumab (AVASTIN), trastuzumab (HERCEPTIN), panitumumab (VECTIBIX), or cetuximab (ERBITUX). Combined administration can include co-administration, either in a single pharmaceutical formulation or using separate formulations, or consecutive administration in either order but generally within a time period such that all active agents can exert their biological activities simultaneously.

Furthermore, treatment with a DLL4 antagonist described herein can include combination treatment with other biologic molecules, such as one or more cytokines (e.g., lymphokines, interleukins, tumor necrosis factors, and/or growth factors) or can be accompanied by surgical removal of tumors, cancer cells, or any other therapy deemed necessary by a treating physician.

In certain embodiments, the treatment involves the administration of a DLL4 antagonist (e.g. an antibody) of the present invention in combination with radiation therapy. Treatment with a DLL4 antagonist can occur prior to, concurrently with, or subsequent to administration of radiation therapy. Dosing schedules for such radiation therapy can be determined by the skilled medical practitioner.

Embodiments of the present disclosure can be further defined by reference to the following non-limiting examples, which describe the use of a DLL4 antagonist for treatment of cancer. It will be apparent to those skilled in the art that many modifications, both to materials and methods, may be practiced without departing from the scope of the present disclosure.

### EXAMPLES

### Example 1

### Phase 1 Study

An open-label Phase .1 dose escalation study of OMP-21M18 in patients with previously treated solid tumors was conducted. There was no remaining standard curative therapy for these patients and no therapy with a demonstrated survival benefit. Prior to enrollment, patients underwent screening to determine study eligibility. The study endpoints included the determination of the safety profile, maximum tolerated dose (MTD), immunogenicity, pharmacokinetics, antitumor activity, and biomarkers of Notch signaling and stem cell-related genes in blood, hair follicles and tumor cells. In the initial phase of the study, dose escalation was performed to determine the maximum tolerated dose. Dose levels of 0.5, 1.0, 2.5, and 5 mg/kg of OMP-21M18 were administered IV weekly for 9 doses and then every other week; and dose levels of 2.5, 5, and 10 mg/kg every other week. No dose escalation or reduction was allowed within a dose cohort. Three patients were treated at each dose level if no dose-limiting toxicities (DLTs) were observed. If 1 of 3 patients experienced a DLT, the dose level was expanded to 6 patients. If 2 or more patients experienced a DLT, no further patients were dosed at that level and 3 additional patients were added to the preceding dose cohort unless 6 patients were being treated at that dose level. Patients were assessed for DLTs from the time of the first dose through 7 days after administration of the 4^{th} dose, but prior to administration of the 5^{th} dose (i.e., Day 28). The MTD was defined as the highest dose level that resulted in less than 2 of 6 subjects experiencing a DLT. The MTD was not reached at 10 mg/kg using a once every other week dosing.

### Pharmacokinetics

The pharmacokinetics of OMP-21M18 in patients participating in the Phase 1 trial were evaluated. Samples from each patient treated with 0.5 mg/kg, 1 mg/kg, 2.5 mg/kg, and 5 mg/kg every week were collected at weekly intervals. Samples from each patient treated with 2.5 mg/kg, 5 mg/kg and 10 mg/kg every other week were collected at weekly intervals. At each time point, approximately 4 mL of blood was collected in a sodium heparin vacutainer tube and centrifuged. The plasma supernatants were collected and frozen at −70° C. until the samples were analyzed.

The level of OMP-21M18 present in the plasma at each time point was quantified and the half-life of OMP-21M18 was calculated. Day 0 and day 49 results from patients dosed on a weekly schedule are shown in Table 1. Day 0 and day 42 results from patients dosed on an every other week schedule are shown in Table 2.

TABLE 1
Noncompartmental Pharmacokinetic
Parameters Every Week Dosing

| | | | | |
|---|---|---|---|---|
| Day | T½ (days) | Cmax (μg/ml) | Cl (ml/hr/kg) | Vss (ml/br) |

0.5 mg/kg

| | | | | |
|---|---|---|---|---|
| 0 | 4.3 | 9 | 0.5 | 86 |
| 49 | 10.4 | 17 | 0.1 | 42 |

1 mg/kg

| | | | | |
|---|---|---|---|---|
| 0 | 5.2 | 27 | 0.4 | 84 |
| 49 | 11.5 | 38 | 0.1 | 49 |

2.5 mg/kg

| | | | | |
|---|---|---|---|---|
| 0 | 4.8 | 62 | 0.4 | 64 |
| 49 | 13.3 | 116 | 0.1 | 36 |

5 mg/kg

| | | | | |
|---|---|---|---|---|
| 0 | 4.3 | 82 | 0.6 | 110 |
| 49 | 12.9 | 231 | 0.1 | 40 |

TABLE 2
Noncompartmental Pharmacokinetic Parameters
Every Other Week Dosing

| | | | | |
|---|---|---|---|---|
| Day | T_{1/2} (days) | Cmax (μg/ml) | Cl (ml/hr/kg) | Vss (ml/hr) |

2.5 mg/kg

| | | | | |
|---|---|---|---|---|
| 0 | 6.7 | 73 | 0.3 | 72 |
| 42 | 7.3 | 108 | 0.1 | 32 |

5 mg/kg

| | | | | |
|---|---|---|---|---|
| 0 | 7.7 | 142 | 0.3 | 82 |
| 42 | 10.2 | 179 | 0.2 | 55 |

10 mg/kg

| | | | | |
|---|---|---|---|---|
| 0 | 9.4 | 258 | 0.3 | 78 |
| 42 | 9.2 | 381 | 0.1 | 41 |

Non-compartmental post-treatment termination half-life was calculated to be 17.1 days. Two compartmental analyses were calculated and showed the alpha half-life to be day, and the beta half-life to be 15 days. The beta half-life of OMP-21M18 in these Phase 1 patients supports a dosing schedule of 2 to 3 weeks.

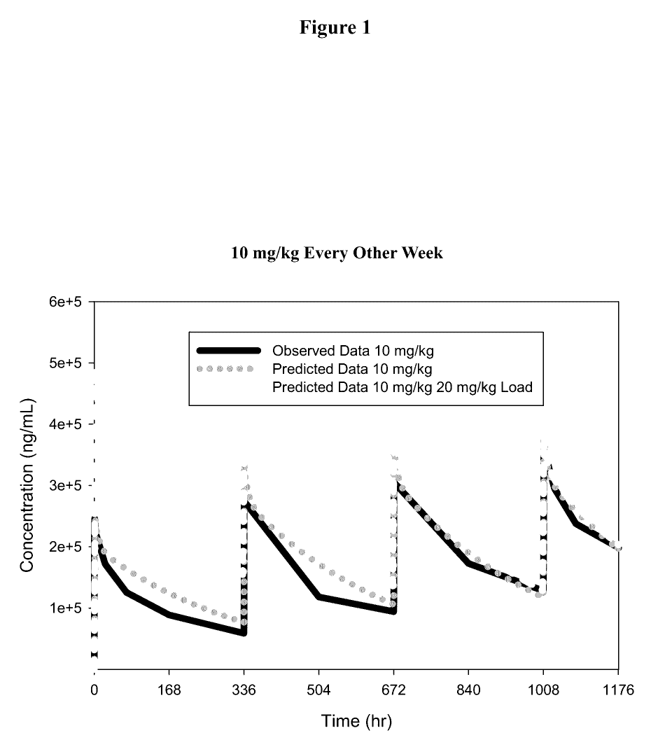

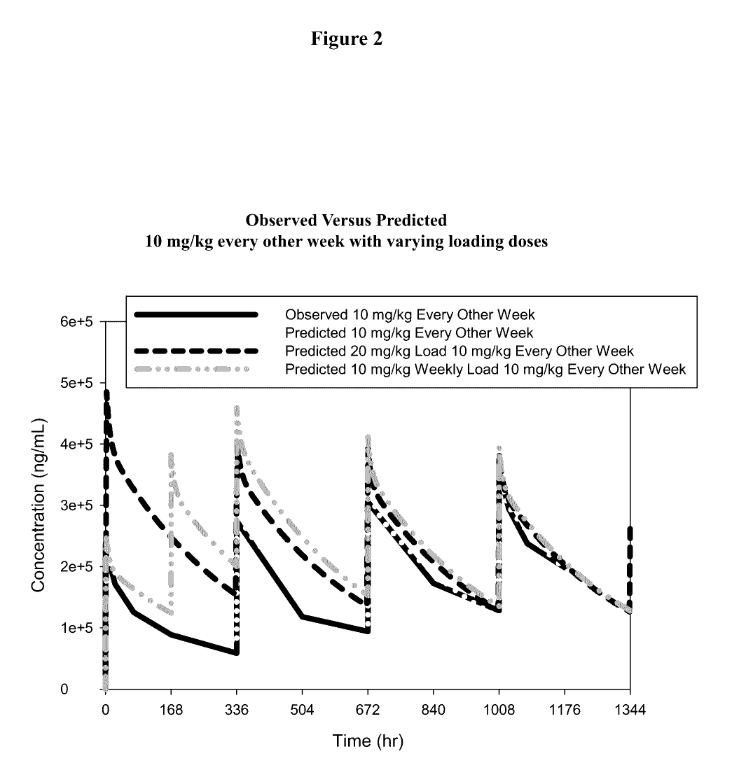

Pharmacokinetic parameters were analyzed at day 1 and day 42 for the regimen of 10 mg/kg every other week. These results were compared to predicted results for the same regimen. In addition, the results were compared to predicted results for a regimen of 10 mg/kg every other week with an initial dose of 20 mg/kg (see Table 3 and FIG. 1). These results were than further compared to a predicted regime of 10 mg/kg initial dose followed by two doses of 10 mg/kg weekly followed by subsequent doses of 10 mg/kg every of week see Table 3 and FIG. 2).

TABLE 3

| | | | | | | | |
|---|---|---|---|---|---|---|---|
| | T_{1/2} | Cmax | Tₗₐₛₜ | Cₗₐₛₜ | AUCₗₐₛₜ | AUC0-∞ | % |
| Regimen | (days) | (μg/ml) | (days) | (μg/ml) | (μg/hr/ml) | (μg/hr/ml) | Extrap |

Day 1 (0-14 days)

| | | | | | | | |
|---|---|---|---|---|---|---|---|
| Observed 10 mg/kg | 10.2 | 246 | 14 | 59 | 34662 | 55406 | 37 |
| every other week | | | | | | | |
| Predicted 10 mg/kg | 10.0 | 242 | 14 | 77 | 43463 | 70130 | 38 |
| every other week | | | | | | | |
| Predicted 10 mg/kg | 10.0 | 485 | 14 | 154 | 86516 | 140254 | 38 |
| every other week with | | | | | | | |
| 20 mg/kg initial dose | | | | | | | |
| Predicted 10 mg/kg | 10.0 | 383 | 14 | 201 | 70883 | 140375 | 50 |
| every other week with | | | | | | | |
| 10 mg/kg dose first 2 | | | | | | | |
| weeks | | | | | | | |

Day 42 (0-14 days)

| | | | | | | | |
|---|---|---|---|---|---|---|---|
| Observed 10 mg/kg | 8.4 | 360 | 49 | 198 | 213515 | 271250 | 21 |
| every other week | | | | | | | |
| Predicted 10 mg/kg | 10.0 | 364 | 56 | 126 | 253959 | 297865 | 15 |
| every other week | | | | | | | |
| Predicted 10 mg/kg | 10.0 | 376 | 56 | 121 | 258458 | 300668 | 14 |
| every other week with | | | | | | | |
| 20 mg/kg initial dose | | | | | | | |
| Predicted 10 mg/kg | NC | NC | NC | NC | NC | NC | NC |
| every other week with | | | | | | | |
| 10 mg/kg dose first 2 | | | | | | | |
| weeks | | | | | | | |

NC = Not calculated

### Assesment of Tumor Burden

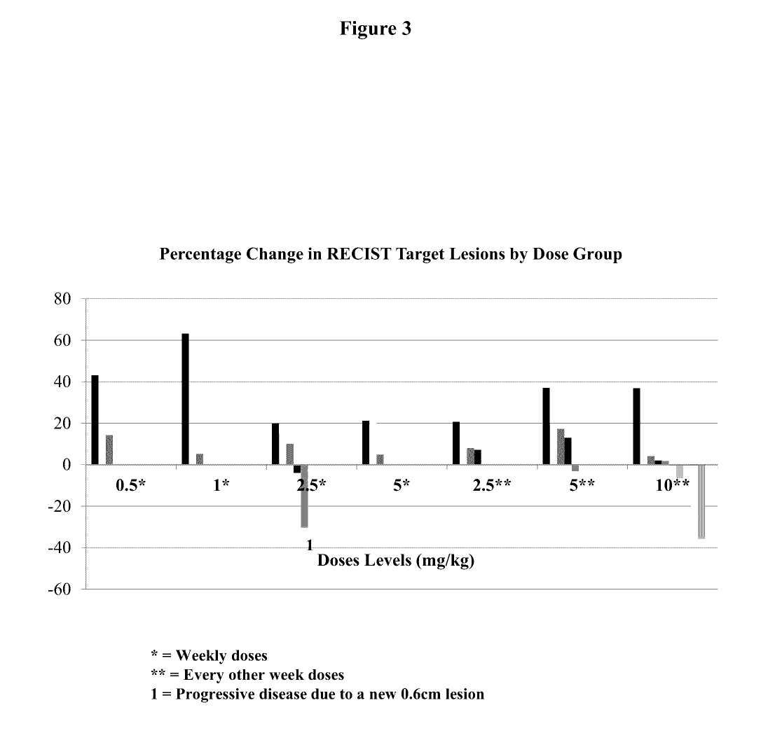

Assessment of the change in tumor burden is an important feature of the clinical evaluation of cancer therapeutics. The RECIST (Response Evaluation Criteria in Solid Tumors) criteria were published in 2000 and updated in 2009 (Eisenhauer et al, 2009, European J Cancer, 45:228-247). The key features of RECIST include definitions of the minimum size of measurable lesions, instructions on how many lesions to follow, and the use of unidimensionai, rather than bidimensional, measures for overall evaluation of tumor burden. The patients in the Phase 1 were evaluated using RECIST criteria. Tumor lesions were measured by CT scan and measured in one dimension and the percentage change in target lesion is shown in FIG. 3. The waterfall plot shows that 8 of 12 patients (67%) receiving OMP-21M18 at a dose of 10 mg/ml had stable disease or a partial response.

### Example 2

### Intermittent Dosing of Anti-DLL4 Antibody in a Pancreatic Xenograft Model and Effect or Tumor Growth

OMP-PN8 pancreatic tumor cells (50,000 cells) were injected subcutaneously into 6-8 week old NOD/SCID mice. Tumors were allowed to grow for 26 days until the average tumor size was approximately 100 mm³. The animals were randomized into turnips (n=10 per group) and treated with anti-DLL4 antibody, gemcitabine, anti-DLL4 antibody in combination with gemcitabine, or a control antibody. The anti-DLL4 antibody was a 1:1 mixture of anti-human DLL4 antibody (21M18) and anti-mouse DLL4 antibody (21R30), with each antibody at 20 mg/kg. Gemcitabine and the control antibody were administered at a dose of 20 mg/kg once a week. The anti-DLL4 antibodies were administered once a week, once every two weeks, or once every four weeks. The agents were administered intraperitoneally. Tumor volumes were measured on the indicated days with electronic calipers.

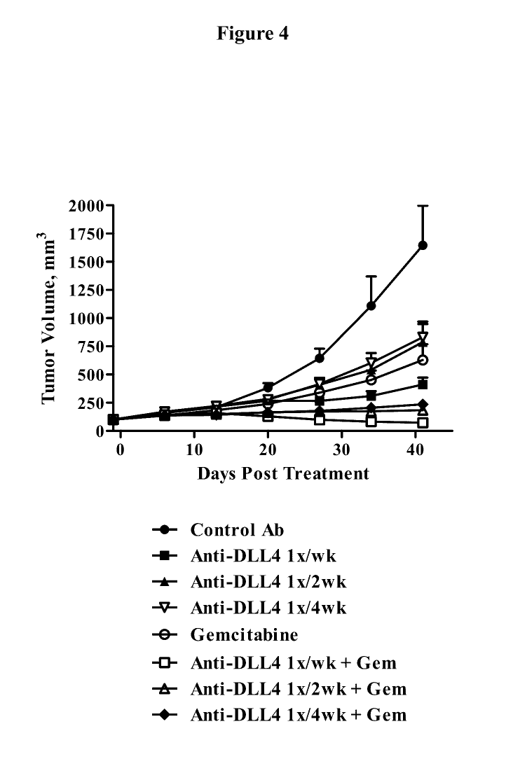

As shown in FIG. 4, when administered as a single agent, a weekly dosing regimen of anti-DLL4 was superior to less frequent dosing. Weekly dosing of anti-DLL4 in combination with gemcitabine was the most efficacious, appearing to completely inhibit tumor growth in this model. Furthermore, less frequent intermittent dosing of anti-DLL4 every two weeks or every four weeks demonstrated tumor growth inhibition at levels almost as great as the weekly regimen. These results demonstrate that the efficacy of anti-DLL4 antibody treatment, especially in combination with a chemotherapeutic agent such as gemcitabine, is maintained with intermittent dosing regimens.

### Example 3

### Intetmittent Dosing of Anti-DLL4 Antibody in a Pancreatic Xenograft Model and Effect on Tumorigenicity

Tumors from mice treated with gemcitabine, the combination of anti-DLL4 antibody and gemcitabine, or control antibody (from the experiment described in Example 2) were harvested and processed to single cell suspensions. The tumor cells were incubated with blotinylated mouse antibodies (α-mouse CD45-biotin 1:100 dilution and rat α-mouse H2Kd-biotin 1:50 dilution, BioLegend, San Diego, Calif.) on ice for 30 min followed by addition or streptavadin-labeled magnetic beads (Invitrogen, Carlsbad, Calif.) to remove mouse cells. The remaining human cells in the suspension were collected, counted, and mixed with a solution of 1:1 (v/v) FACS buffer and Matriael, 90 cells per mouse were injected subcutaneously in NOD/SCID mice (n=10 mice per group). Tumors were allowed to grow in the recipient mice without treatment until day 64.

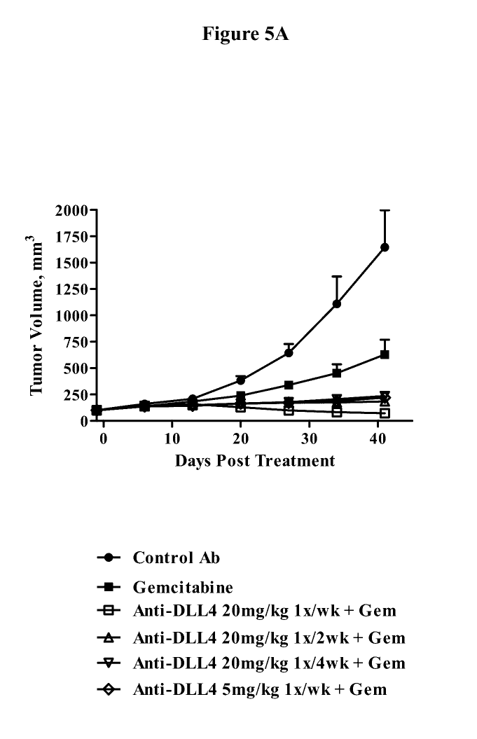

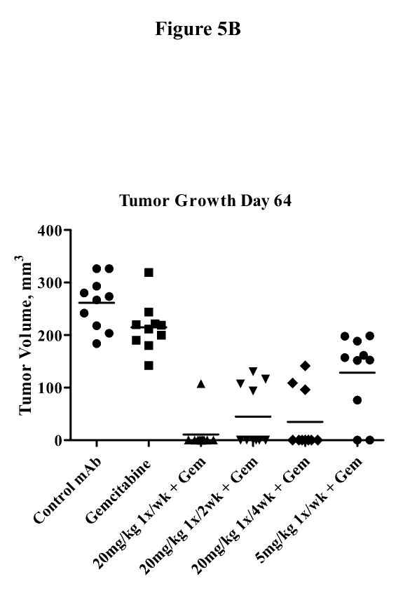

FIG. 5A shows a subset of the results from FIG. 4, as well as including results from a group of mice treated weekly with a combination of gemcitabine and anti-DLL4 at 5 mg/kg of 21M18 and 5 mg/kg of 21R30. The combination of gemcitabine and the loser dose of anti-DLL4 inhibited tumor growth at a level similar to treatment with the higher amount of anti-DLL4. When assessing the tumorigenicity of the tumor cells from these treated animals, it was demonstrated that weekly treatment with the combination of gemcitabine and anti-DLL4 at 20 mg/kg greatly reduced the tumorigenicity of the OMP-PN8 tumor cells (FIG. 5B). In fact only 1 mouse out of the group of ten developed a tumor from the cells from these treated animals. Furthermore, intermittent dosing with anti-DLL4 once every two weeks or once every four weeks was also effective as shown by reduced frequency of tumor growth compared to the control antibody or gemcitabine alone groups. Although weekly treatment with a combination of gemcitabine and 5 mg/kg anti-DLL4 was able to strongly inhibit tumor growth, this treatment was less effective in reducing tumorigenicity than treatment with higher doses of anti-DLL4, Paralleling the results of Example 2, this study demonstrated that the efficacy of anti-DLL4 antibody treatment, especially in combination with a chemotherapeutic agent such as gemcitabine, is maintained with intermittent dosing regimens. Importantly, not only is tumor growth inhibited with intermittent dosing regimens, but the tumorigenicity of tumor cells is greatly reduced using intermittent dosing regimens.

### Example 4

### Intermittent Dosing; of Anti-DLL4 Antibody in a Pancreatic Xenograft Recurrence Model and Effect on Tumor Growth

OMP-8 pancreatic tumor cells (50,000 cells) were injected subcutaneously into 6-8 week old NOD/SCID mice. Tumors were allowed to grow for 30 days until the average tumor size was approximately 140 mm³. The animals were randomized into groups (n=10 per group) and were treated with a combination of gemcitabine at 70 mg/kg and control antibody at 20 mg/kg once a week, or with a combination of gemcitabine at 70 mg/kg and anti-DLL4 (mixture of 21M18 and 21R30 as described above) at either 20 mg/kg once a week, 20 mg/kg once every 2 weeks, or 20 mg/kg once every 4 weeks, or anti-DLL4 antibody at 5 mg/kg once a week. Combination treatment was administered for four weeks, after which the gemcitabine treatments were stopped and the antibody treatments continued for the duration of the experiment. The agents were administered intraperitoneally. Tumor volumes were measured on the indicated days with electronic calipers.

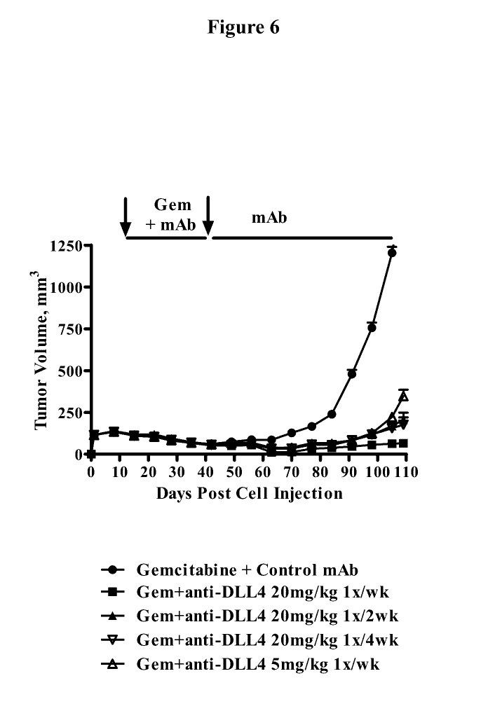

As shown in FIG. 6, when gemcitabine treatment was discounted, tumor recurrence was relatively rapid and resulting tumor volumes were large. In contrast, in all dosing regimens additional anti-DLL4 antibody treatment was effective in suppressing and/or delaying tumor recurrence after gemcitabine treatment was discontinued. Weekly dosing at 20 mg/kg was the most effective at inhibiting tumor recurrence. Intermittent dosing with anti-DLL4 at 20 mg/kg once every 2 weeks and once every 4 weeks was also able to inhibit tumor growth, while weekly dosing at 5 mg/kg was somewhat less effective. Thus, intermittent dosing of anti-DLL4 antibody treatment even after treatment with gemcitabine had been discontinued was effective in delaying tumor recurrence.

### Example 5

### Effect of Intermittent Dosing of Anti-DLL4 Antibody on Mouse Liver Toxicity

DLL4 blockade has been reported to produce histopathologic changes in the liver, including sinusoidal dilation and hepatocyte atrophy. Therefore, at the end of the dosing phase of the experiment described in Example 2, livers were harvested from the mice in the various treatment groups and evaluated for pathologic changes. The livers were fixed in formalin, sectioned, and stained with hematoxylin and eosin (H&E). Photomicrographs were reviewed at low power and at higher magnifications. Livers from the mice treated with 20 mg/kg anti-DLL4 antibody once a week showed sinusoidal dilation and an irregular surface. These effects were also apparent, although less severe, in the livers from the mice treated with 5 mg/kg antinDLL4 once a week. In contrast, the livers from mice treated with 20 mg/kg anti-DLL4 once every 4 weeks were similar to livers from mice treated with a control antibody. These results demonstrated that anti-DLL4 antibody treatment appeared to have less toxicity when used in an intermittent dosing regimen than when used on a weekly basis. Thus, less frequent administration of an anti-DLL4 antibody may be associated with a better therapeutic index.

It is understood that the examples and embodiments described herein are for illustrative purposes only and that various modifications or changes in light thereof will be suggested to persons skilled in the art and are to be included within the spirit and purview of this application.

All publications, patents, and patent applications cited herein are hereby incorporated by reference in their entirety for all purposes to the same extent as if each individual publication, patent or patent application were specifically and individually indicated to be so incorporated by reference.

Heavy-chain CDR1
SEQ ID NO: 1
TAYYIH
Heavy chain CDR2, H2
SEQ ID NO: 2
YISCYNGATNYNQKFKG
Heavy Chain CDR2, H7
SEQ ID NO: 3
YISSYNGATNYNQKFKG
Heavy chain CDR2, H9
SEQ ID NO: 4
YISVYNGATNYNQKFKG
Heavy chain CDR3
SEQ ID NO: 5
RDYDYDVGMDY
Heavy chain variable region without signal
sequence, H7
SEQ ID NO: 6
QVQLVQSGAEVKKPGASVKISCKASGYSFTAYYIHWVKQAPGQGLEWIGY
ISSYNGATNYNQKFKGRVTFTTDTSTSTAYMELRSLRSDDTAVYYCARDY
DYDVGMDYWGQGTLVTVSS
Heavy chain variable region without signal
sequence, H2
SEQ ID NO: 7
QVQLVQSGAEVKKPGASVKISCKASGYSFTAYYIHWVKQAPGQGLEWIGY
ISCYNGATNYNQKFKGRVTFTTDTSTSTAYMELRSLRSDDTAVYYCARDY
DYDVGMDYWGQGTLVTVSS
Heavy chain variable region without signal
sequence, H9
SEQ ID NO: 8
QVQLVQSGAEVKKPGASVKISCKASGYSFTAYYIHWVKQAPGQGLEWIGY
ISVYNGATNYNQKFKGRVTFTTDTSTSTAYMELRSLRSDDTAVYYCARDY
DYDVGMDYWGQGTLVTVSS
Light chain CDR1
SEQ ID NO: 9
RASESVDNYGISFMK
Light chain CDR2
SEQ ID NO: 10
AASNQGS
Light chain CDR3
SEQ ID NO: 11
QQSKEVPWTFGG
Light chain variable region without signal
sequence
SEQ ID NO :12
DIVMTQSPDSLAVSLGERATISCRASESVDNYGISFMKWFQQKPGQPPKL
LIYAASNQGSGVPDRFSGSGSGTDFTLTISSLQAEDVAVYYCQQSKEVPW
TFGGGTKVEIK
Human DLL4 Extracellular Domain (with putative
signal sequence underlined)
SEQ ID NO: 13
MAAASRSASGWALLLLVALWQQRAAGSGVFQLQLQEFINERGVLASGRPC
EPGCRTFFRVCLKHFQAVVSPGPCTFGTVSTPVLGTNSFAVRDDSSGGGR
NPLQLPFNFTWPGTFSLIIEAWHAPGDDLRPEALPPDALISKIAIQGSLA
VGQNWLLDEQTSTLTRLRYSYRVICSDNYYGDNCSRLCKKRNDHFGHYVC
QPDGNLSCLPGWTGEYCQQPICLSGCHEQNGyCSKPAECLCRPGWQGRLC
NECIPHNGCRHGTCSTPWQCTCDEGWGGLFCDQDLNYCTHHSPCKNGATC
SNSGQRSYTCTCRPGYTGVDCELELSECDSNPCRNGGSCKDQEDGYHCLC
PPGYYGLHCEHSTLSCADSPCFNGGSCRERNQGANYACECPPNFTGSNCE
KKVDRCTSNPCANGGQCLNRGPSRMCRCRPGFTGTYCELHVSDCARNPCA
HGGTCHDLENGLMCTCPAGFSGRRCEVRTSIDACASSPCFNRATCYTDLS
TDTFVCNCPYGFVGSRCEFPVG
Amino acids 27-217 of Human DLL4 Extracellular
Domain (withoul putative signal sequence)
SEQ ID NO: 14
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYC
Human DLL4 N-Terminal Region (with putative signal
sequence underlined)
SEQ ID NO: 15
MAAASRSASGWALLLLVALWQQRAAGSGVFQLQLQEFINERGVLASGRPC
EPGCRTFFRVCLKHFQAVVSPGPCTFGTVSTPVLGTNSFAVRDDSSGGGR
NPLQLPFNFTWPGTFSLIIEAWHAPGDDLRPEALPPDALISKIAIOGSLA
VGQN
Human DLL4 DSL Region
SEQ ID NO: 16
WLLDEQTSTLTRLRYSYRVICSDNYYGDNCSRLCKKRNDHFGHYVCQPDG
NLSCLPGWTGEYC
Amino acids 66-73 of Human DLL4
SEQ ID NO: 17
QAWSPGP
Amino acids 139-146 of Human DLL4
SEQ ID NO: 18
LISKIAIQ
FLAG peptide
SEQ ID NO: 19
DYKDDDDK

1-18. (canceled)

19. A method for treating pancreatic cancer in a human patient comprising:
administering a delta-like ligand 4 (DLL4) antagonist in combination with at least gemcitabine and a taxane,
wherein the DLL4 antagonist is administered to the patient once every 2 weeks, once every 3 weeks, or once every 4 weeks at a dose of about 2 mg/kg to about 10 mg/kg, and
wherein the DLL4 antagonist is an antibody comprising a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

20. The method of claim 19, wherein the DLL4 antagonist is administered once every 2 weeks.

21. The method of claim 19, wherein the DLL4 antagonist is administered once every 3 weeks.

22. The method of claim 19, wherein the dose of the DLL4 antagonist is about 2.5 mg/kg to about 5 mg/kg.

23. The method of claim 19, wherein the dose of the DLL4 antagonist is about 5 mg/kg to about 7.5 mg/kg.

24. The method of claim 19, wherein the DLL4 antagonist comprises a heavy chain variable region comprising the amino acids of SEQ ID NO:6 and a light chain variable region comprising the amino acids of SEQ ID NO:12.

25. The method of claim 19, wherein the DLL4 antagonist comprises the same heavy and light chain variable region amino acid sequences as an antibody encoded by a plasmid deposited with ATCC having deposit no. PTA-8425.

26. The method of claim 19, wherein the DLL4 antagonist is encoded by the plasmid having ATCC deposit no. PTA-8425.

27. The method of claim 19, wherein the taxane is paclitaxel.

28. The method of claim 19, wherein the taxane is albumin-bound paclitaxel (ABRAXANE®).

29. A method for treating pancreatic cancer in a human patient comprising:
administering a delta-like ligand 4 (DLL4) antagonist in combination with gemcitabine and a taxane,
wherein the DLL4 antagonist is administered to the patient once every 2 weeks at a dose of about 2.5 mg/kg to about 5 mg/kg, and
wherein the DLL4 antagonist is an antibody comprising a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5), and a light chain CDR1 comprising RASESVDNYGISFMK (SEQ ID NO:9), a light chain CDR2 comprising AASNQGS (SEQ ID NO:10), and a light chain CDR3 comprising QQSKEVPWTFGG (SEQ ID NO:11).

30. The method of claim 29, wherein the taxane is paclitaxel.

31. The method of claim 29, wherein the taxane is albumin-bound paclitaxel (ABRAXANE®).

32. A method for treating cancer in a human patient comprising:
administering a delta-like ligand 4 (DLL4) antagonist bispecific antibody in combination with at least one additional chemotherapeutic agent,
wherein the DLL4 antagonist bispecific antibody is administered to the patient once every 2 weeks, once every 3 weeks, or once every 4 weeks at a dose of about 2 mg/kg to about 10 mg/kg, and
wherein the DLL4 antagonist bispecific antibody comprises a heavy chain variable region having at least 95% sequence identity to SEQ ID NO:6 and a light chain variable region having at least 95% identity to SEQ ID NO:12.

33. The method of claim 32, wherein the DLL4 antagonist bispecific antibody is administered once every 2 weeks.

34. The method of claim 32, wherein the DLL4 antagonist bispecific antibody is administered once every 3 weeks.

35. The method of claim 32, wherein the DLL4 antagonist bispecific antibody is administered once every 4 weeks.

36. The method of claim 32, wherein the DLL4 antagonist bispecific antibody specifically binds DLL4 and VEGF.

37. The method of claim 32, wherein the DLL4 antagonist bispecific antibody is administered in combination with gemcitabine and a taxane.

38. The method of claim 37, wherein the taxane is paclitaxel.

39. The method of claim 38, wherein the taxane is albumin-bound paclitaxel (ABRAXANE®).
